(19) United States
(12) Patent Application Publication
(10) Pub. No.: US 2020/0199690 A1
(43) Pub. Date: Jun. 25, 2020

(54) IDENTIFICATION OF PATIENTS IN NEED OF PD-L1 INHIBITOR COTHERAPY

(71) Applicant: Hoffmann-La Roche Inc., Little Falls, NJ (US)

(72) Inventors: Anton BELOUSOV, Penzberg (DE); Giampaolo Bianchini, Bergamo (IT); Luca GIANNI, Milano (IT); Marlene THOMAS, Rheinfelden (DE)

(73) Assignee: Hoffmann-La Roche Inc., Little Falls, NJ (US)

(21) Appl. No.: 16/814,688

(22) Filed: Mar. 10, 2020

Related U.S. Application Data
(63) Continuation of application No. 15/815,384, filed on Nov. 16, 2017.
Application No. 15/815,384 is a continuation of application No. 14/720,643, filed on May 22, 2015, now abandoned.
Application No. 14/720,643 is a continuation of application No. PCT/EP2013/075162, filed on Nov. 29, 2013.

(30) Foreign Application Priority Data
Nov. 30, 2012 (EP) 12195182.6
Dec. 7, 2012 (EP) 12196177.5

(51) Int. Cl. C12Q 1/6886; G01N 33/68; G01N 33/574; A61K 39/395; A61K 31/337; G01N 33/74

(52) U.S. Cl. CPC C12Q 1/6886; G01N 33/6866; G01N 33/57415; A61K 39/39558; A61K 31/337; G01N 33/743; A61K 2039/505; G01N 2333/723; G01N 2333/70532; G01N 2333/57; C12Q 2600/158; C12Q 2600/106; G01N 33/6872

(57) ABSTRACT

The present invention relates to means and methods for determining whether a patient is in need of a PD-L1 inhibitor cotherapy. A patient is determined to be in need of the PD-L1 inhibitor cotherapy if a low or absent ER expression level and an expression level of programmed death ligand 1 (PD-L1) that is increased in comparison to a control is measured in vitro in a sample from the patient. The patient is undergoing therapy comprising a modulator of the HER2/neu (ErbB2) signaling pathway (like Trastuzumab) and a chemotherapeutic agent (like dodetaxel) or such a therapy is contemplated for the patient. Also provided herein are means and methods for treating a cancer in a cancer patient for whom therapy comprising a modulator of the HER2/neu (ErbB2) signaling pathway (like Trastuzumab) and a chemotherapeutic agent (like dodetaxel) is contemplated, wherein the patient is to receive PD-L1 inhibitor cotherapy.

### CROSS-REFERENCE RELATED APPLICATIONS

This application is a continuation of U.S. patent application Ser. No. 15/815,384, filed Nov. 16, 2017, which is a continuation of U.S. patent application Ser. No. 14/720,643, filed May 22, 2015, which is a continuation of International Patent Application No. PCT/EP2013/075162, filed Nov. 29, 2013, which claims priority to European Patent Application No. 12195182.6, filed Nov. 30, 2012 and European Patent Application No. 12196177.5, filed Dec. 7, 2012, the disclosures of each of which are incorporated by reference herein in their entireties.

### SEQUENCE LISTING

The instant application contains a Sequence Listing submitted via EFS-Web and hereby incorporated by reference in its entirety. Said ASCII copy, created on Mar. 9, 2020, is named P31286-US-2_Sequence_Listing.txt, and is 782,293 bytes in size.

The present invention relates to means and methods for determining whether a patient is in need of a PD-L1 inhibitor cotherapy. A patient is determined to be in need of the PD-L1 inhibitor cotherapy if a low or absent ER expression level and an expression level of programmed death ligand 1 (PD-L1) that is increased in comparison to a control is measured in vitro in a sample from the patient. The patient is undergoing therapy comprising a modulator of the HER2/neu (ErbB2) signaling pathway (like Trastuzumab) and a chemotherapeutic agent (like dodetaxel) or such a therapy is contemplated for the patient. Also provided herein are means and methods for treating a cancer in a cancer patient for whom therapy comprising a modulator of the HER2/neu (ErbB2) signaling pathway (like Trastuzumab) and a chemotherapeutic agent (like dodetaxel) is contemplated, wherein the patient is to receive PD-L1 inhibitor cotherapy.

The HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4 or tyro2).

EGFR, encoded by the erbB1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-α), by the same tumor cells resulting in receptor activation by an autocrine stimulatory pathway. Baselga and Mendelsohn Pharmac. Ther. 64:127-154 (1994). Monoclonal antibodies directed against the EGFR or its ligands, TGF-α and EGF, have been evaluated as therapeutic agents in the treatment of such malignancies. See, e.g., Baselga and Mendelsohn., supra; Masui et al. Cancer Research 44:1002-1007 (1984); and Wu et al. J. Clin. Invest. 95:1897-1905 (1995).

The second member of the HER family, p185^{neu}, was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis (Slamon et al., Science, 235:177-182 (1987); Slamon et al., Science, 244:707-712 (1989); and U.S. Pat. No. 4,968,603). To date, no point mutation analogous to that in the neu proto-oncogene has been reported for human tumors. Overexpression of HER2 (frequently but not uniformly due to gene amplification) has also been observed in other carcinomas including carcinomas of the stomach, endometrium, salivary gland, lung, kidney, colon, thyroid, pancreas and bladder. See, among others, King et al., Science, 229:974 (1985); Yokota et al., Lancet: 1:765-767 (1986); Fukushige et al., Mol Cell Biol., 6:955-958 (1986); Guerin et al., Oncogene Res., 3:21-31 (1988); Cohen et al., Oncogene, 4:81-88 (1989); Yonemura et al., Cancer Res., 51:1034 (1991); Borst et al., Gynecol. Oncol., 38:364 (1990); Weiner et al., Cancer Res., 50:421-425 (1990); Kern et al., Cancer Res., 50:5184 (1990); Park et al., Cancer Res., 49:6605 (1989); Zhau et al., Mol. Carcinog., 3:254-257 (1990); Aasland et al. Br. J. Cancer 57:358-363 (1988); Williams et al. Pathobiology 59:46-52 (1991); and McCann et al., Cancer, 65:88-92 (1990). HER2 may be overexpressed in prostate cancer (Gu et al. Cancer Lett. 99:185-9 (1996); Ross et al. Hum. Pathol. 28:827-33 (1997); Ross et al. Cancer 79:2162-70 (1997); and Sadasivan et al. J Urol. 150:126-31 (1993)).

Antibodies directed against the rat p185^{neu} and human HER2 protein products have been described. Drebin and colleagues have raised antibodies against the rat neu gene product, p185^{neu}. See, for example, Drebin et al., Cell 41:695-706 (1985); Myers et al., Meth. Enzym. 198:277-290 (1991); and WO94/22478. Drebin et al. Oncogene 2:273-277 (1988) report that mixtures of antibodies reactive with two distinct regions of p185^{neu} result in synergistic anti-tumor effects on neu-transformed NIH-3T3 cells implanted into nude mice. See also U.S. Pat. No. 5,824,311 issued Oct. 20, 1998.

Hudziak et al., Mol. Cell. Biol. 9(3):1165-1172 (1989) describe the generation of a panel of HER2 antibodies which were characterized using the human breast tumor cell line SK-BR-3. Relative cell proliferation of the SK-BR-3 cells following exposure to the antibodies was determined by crystal violet staining of the monolayers after 72 hours. Using this assay, maximum inhibition was obtained with the antibody called 4D5 which inhibited cellular proliferation by 56%. Other antibodies in the panel reduced cellular proliferation to a lesser extent in this assay. The antibody 4D5 was further found to sensitize HER2-overexpressing breast tumor cell lines to the cytotoxic effects of TNF-α. See also U.S. Pat. No. 5,677,171 issued Oct. 14, 1997. The HER2 antibodies discussed in Hudziak et al. are further characterized in Fendly et al. Cancer Research 50:1550-1558 (1990); Kotts et al. In Vitro 26(3):59A (1990); Sarup et al. Growth Regulation 1:72-82 (1991); Shepard et al. J. Clin. Immunol. 11(3):117-127 (1991); Kumar et al. Mol. Cell. Biol. 11(2):979-986 (1991); Lewis et al. Cancer Immunol. Immunother. 37:255-263 (1993); Pietras et al. Oncogene 9:1829-1838 (1994); Vitetta et al. Cancer Research 54:5301-5309 (1994); Sliwkowski et al. J. Biol. Chem. 269(20):14661-14665 (1994); Scott et al. J. Biol. Chem. 266:14300-5 (1991); D'souza et al. Proc. Natl. Acad. Sci. 91:7202-7206 (1994); Lewis et al. Cancer Research 56:1457-1465 (1996); and Schaefer et al. Oncogene 15:1385-1394 (1997).

A recombinant humanized version of the murine HER2 antibody 4D5 (huMAb4D5-8, rhuMAb HER2, Trastuzumab or Herceptin™; U.S. Pat. No. 5,821,337) is clinically active in patients with HER2-overexpressing metastatic breast cancers that have received extensive prior anti-cancer therapy (Baselga et al., J Clin. Oncol. 14:737-744 (1996)). Trastuzumab received marketing approval from the Food and Drug Administration Sep. 25, 1998 for the treatment of patients with metastatic breast cancer whose tumors overexpress the HER2 protein.

Humanized anti-ErbB2 antibodies include huMAb4D5-1, huMAb4D5-2, huMAb4D5-3, huMAb4D5-4, huMAb4D5-5, huMAb4D5-6, huMAb4D5-7 and huMAb4D5-8 (HERCEPTIN®) as described in Table 3 of U.S. Pat. No. 5,821,337 expressly incorporated herein by reference; humanized 520C9 (WO 93/21319) and humanized 2C4 antibodies as described in WO 01/000245 expressly incorporated herein by reference.

Pertuzumab (see e.g. WO 01/000245) is the first of a new class of agents known as HER dimerization inhibitors (HDIs). Pertuzumab binds to HER2 at its dimerization domain, thereby inhibiting its ability to form active dimer receptor complexes and thus blocking the downstream signal cascade that ultimately results in cell growth and division (see Franklin, M. C., Cancer Cell 5 (2004) 317-328). Pertuzumab is a fully humanized recombinant monoclonal antibody directed against the extracellular domain of HER2. Binding of Pertuzumab to the HER2 on human epithelial cells prevents HER2 from forming complexes with other members of the HER family (including EGFR, HER3, HER4) and probably also HER2 homodimerization. By blocking complex formation, Pertuzumab prevents the growth stimulatory effects and cell survival signals activated by ligands of HER1, HER3 and HER4 (e.g. EGF, TGFalpha, amphiregulin, and the heregulins). Another name for Pertuzumab is 2C4. Pertuzumab is a fully humanized recombinant monoclonal antibody based on the human IgG1(K) framework sequences. The structure of Pertuzumab consists of two heavy chains (449 residues) and two light chains (214 residues). Compared to Trastuzumab (Herceptin®), Pertuzumab has 12 amino acid differences in the light chain and 29 amino acid differences in the IgG1 heavy chain.

Other HER2 antibodies with various properties have been described in Tagliabue et al. Int. J. Cancer 47:933-937 (1991); McKenzie et al. Oncogene 4:543-548 (1989); Maier et al. Cancer Res. 51:5361-5369 (1991); Bacus et al. Molecular Carcinogenesis 3:350-362 (1990); Stancovski et al. PNAS (USA) 88:8691-8695 (1991); Bacus et al. Cancer Research 52:2580-2589 (1992); Xu et al. Int. J. Cancer 53:401-408 (1993); WO94/00136; Kasprzyk et al. Cancer Research 52:2771-2776 (1992); Hancock et al. Cancer Res. 51:4575-4580 (1991); Shawver et al. Cancer Res. 54:1367-1373 (1994); Arteaga et al. Cancer Res. 54:3758-3765 (1994); Harwerth et al. J. Biol. Chem. 267:15160-15167 (1992); U.S. Pat. No. 5,783,186; and Klapper et al. Oncogene 14:2099-2109 (1997).

Homology screening has resulted in the identification of two other HER receptor family members; HER3 (U.S. Pat. Nos. 5,183,884 and 5,480,968 as well as Kraus et al. PNAS (USA) 86:9193-9197 (1989)) and HER4 (EP Pat. Appln. No 599,274; Plowman et al., Proc. Natl. Acad. Sci. USA, 90:1746-1750 (1993); and Plowman et al., Nature, 366:473-475 (1993)). Both of these receptors display increased expression on at least some breast cancer cell lines.

The HER receptors are generally found in various combinations in cells and heterodimerization is thought to increase the diversity of cellular responses to a variety of HER ligands (Earp et al. Breast Cancer Research and Treatment 35: 115-132 (1995)). EGFR is bound by six different ligands; epidermal growth factor (EGF), transforming growth factor alpha (TGF-α), amphiregulin, heparin binding epidermal growth factor (HB-EGF), betacellulin and epiregulin (Groenen et al. Growth Factors 11:235-257 (1994)). A family of heregulin proteins resulting from alternative splicing of a single gene are ligands for HER3 and HER4. The heregulin family includes alpha, beta and gamma heregulins (Holmes et al., Science, 256:1205-1210 (1992); U.S. Pat. No. 5,641,869; and Schaefer et al. Oncogene 15:1385-1394 (1997)); neu differentiation factors (NDFs), glial growth factors (GGFs); acetylcholine receptor inducing activity (ARIA); and sensory and motor neuron derived factor (SMDF). For a review, see Groenen et al. Growth Factors 11:235-257 (1994); Lemke, G. Molec. & Cell. Neurosci. 7:247-262 (1996) and Lee et al. Pharm. Rev. 47:51-85 (1995). Recently three additional HER ligands were identified; neuregulin-2 (NRG-2) which is reported to bind either HER3 or HER4 (Chang et al. Nature 387 509-512 (1997); and Carraway et al Nature 387:512-516 (1997)); neuregulin-3 which binds HER4 (Zhang et al. PNAS (USA) 94(18):9562-7 (1997)); and neuregulin-4 which binds HER4 (Harari et al. Oncogene 18:2681-89 (1999)) HB-EGF, betacellulin and epiregulin also bind to HER4.

While EGF and TGFα do not bind HER2, EGF stimulates EGFR and HER2 to form a heterodimer, which activates EGFR and results in transphosphorylation of HER2 in the heterodimer. Dimerization and/or transphosphorylation appears to activate the HER2 tyrosine kinase. See Earp et al., supra. Likewise, when HER3 is co-expressed with HER2, an active signaling complex is formed and antibodies directed against HER2 are capable of disrupting this complex (Sliwkowski et al., J Biol. Chem., 269(20):14661-14665 (1994)). Additionally, the affinity of HER3 for heregulin (HRG) is increased to a higher affinity state when co-expressed with HER2. See also, Levi et al., Journal of Neuroscience 15: 1329-1340 (1995); Morrissey et al., Proc. Natl. Acad. Sci. USA 92: 1431-1435 (1995); and Lewis et al., Cancer Res., 56:1457-1465 (1996) with respect to the HER2-HER3 protein complex. HER4, like HER3, forms an active signaling complex with HER2 (Carraway and Cantley, Cell 78:5-8 (1994)).

Also, antibody variant compositions are described in the art. U.S. Pat. No. 6,339,142 describes a HER2 antibody composition comprising a mixture of anti-HER2 antibody and one or more acidic variants thereof, wherein the amount of the acidic variant(s) is less than about 25%. Trastuzumab is the exemplified HER2 antibody. Reid et al. Poster presented at Well Characterized Biotech Pharmaceuticals conference (January, 2003) “Effects of Cell Culture Process Changes on Humanized Antibody Characteristics” describes an unnamed, humanized IgG1 antibody composition with N-terminal heterogeneities due to combinations of VHS signal peptide, N-terminal glutamine, and pyroglutamic acid on the heavy chain thereof. Harris et al. “The Ideal Chromatographic Antibody Characterization Method” talk presented at the IBC Antibody Production Conference (February, 2002) reports a VHS extension on the heavy chain of E25, a humanized anti-IgE antibody. Rouse et al. Poster presented at WCBP “Glycoprotein Characterization by High Resolution Mass Spectrometry and Its Application to Biopharmaceutical Development” (Jan. 6-9, 2004) describes a monoclonal antibody composition with N-terminal heterogeneity resulting from AHS or HS signal peptide residues on the light chain thereof. In a presentation at IBC Meeting (September, 2000) “Strategic Use of Comparability Studies and Assays for Well Characterized Biologicals,” Jill Porter discussed a late-eluting form of ZENAPAX™ with three extra amino acid residues on the heavy chain thereof. US2006/0018899 describes a composition comprising a main species pertuzumab antibody and an amino-terminal leader extension variant, as well as other variant forms of the pertuzumab antibody.

Patent publications related to HER antibodies include: U.S. Pat. Nos. 5,677,171, 5,720,937, 5,720,954, 5,725,856, 5,770,195, 5,772,997, 6,165,464, 6,387,371, 6,399,063, US2002/0192211A1, U.S. Pat. Nos. 6,015,567, 6,333,169, 4,968,603, 5,821,337, 6,054,297, 6,407,213, 6,719,971, 6,800,738, US2004/0236078A1, U.S. Pat. Nos. 5,648,237, 6,267,958, 6,685,940, 6,821,515, WO98/17797, U.S. Pat. Nos. 6,127,526, 6,333,398, 6,797,814, 6,339,142, 6,417,335, 6,489,447, WO99/31140, US2003/0147884A1, US2003/0170234A1, US2005/0002928A1, U.S. Pat. No. 6,573,043, US2003/0152987A1, WO99/48527, US2002/0141993A1, WO01/00245, US2003/0086924, US2004/0013667A1, WO00/69460, WO01/00238, WO01/15730, U.S. Pat. No. 6,627,196B1, U.S. Pat. No. 6,632,979B1, WO01/00244, US2002/0090662A1, WO01/89566, US2002/0064785, US2003/0134344, WO 04/24866, US2004/0082047, US2003/0175845A1, WO03/087131, US2003/0228663, WO2004/008099A2, US2004/0106161, WO2004/048525, US2004/0258685A1, U.S. Pat. Nos. 5,985,553, 5,747,261, 4,935,341, 5,401,638, 5,604,107, WO 87/07646, WO 89/10412, WO 91/05264, EP 412,116 B1, EP 494,135 B1, U.S. Pat. No. 5,824,311, EP 444,181 B1, EP 1,006,194 A2, US 2002/0155527A1, WO 91/02062, U.S. Pat. Nos. 5,571,894, 5,939,531, EP 502,812 B1, WO 93/03741, EP 554,441 B1, EP 656,367 A1, U.S. Pat. Nos. 5,288,477, 5,514,554, 5,587,458, WO 93/12220, WO 93/16185, U.S. Pat. No. 5,877,305, WO 93/21319, WO 93/21232, U.S. Pat. No. 5,856,089, WO 94/22478, U.S. Pat. Nos. 5,910,486, 6,028,059, WO 96/07321, U.S. Pat. Nos. 5,804,396, 5,846,749, EP 711,565, WO 96/16673, U.S. Pat. Nos. 5,783,404, 5,977,322, 6,512,097, WO 97/00271, U.S. Pat. Nos. 6,270,765, 6,395,272, 5,837,243, WO 96/40789, U.S. Pat. Nos. 5,783,186, 6,458,356, WO 97/20858, WO 97/38731, U.S. Pat. Nos. 6,214,388, 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, WO02/05791, WO 02/11677, U.S. Pat. No. 6,582,919, US2002/0192652A1, US 2003/0211530A1, WO 02/44413, US 2002/0142328, U.S. Pat. No. 6,602,670 B2, WO 02/45653, WO 02/055106, US 2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. Nos. 5,705,157, 6,123,939, EP 616,812 B1, US 2003/0103973, US 2003/0108545, U.S. Pat. No. 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842 and WO 03/86467.

Patients treated with the HER2 antibody Trastuzumab/Herceptin™ are selected for therapy based on HER2 protein overexpression/gene amplification; see, for example, WO99/31140 (Paton et al.), US2003/0170234A1 (Hellmann, S.), and US2003/0147884 (Paton et al.); as well as WO01/89566, US2002/0064785, and US2003/0134344 (Mass et al.). See, also, US2003/0152987, Cohen et al., concerning immunohistochemistry (IHC) and fluorescence in situ hybridization (FISH) for detecting HER2 overexpression and amplification. WO2004/053497 and US2004/024815A1 (Bacus et al.), as well as US 2003/0190689 (Crosby and Smith), refer to determining or predicting response to Trastuzumab therapy. US2004/013297A1 (Bacus et al.) concerns determining or predicting response to ABX0303 EGFR antibody therapy. WO2004/000094 (Bacus et al.) is directed to determining response to GW572016, a small molecule, EGFR-HER2 tyrosine kinase inhibitor. WO2004/063709, Amler et al., refers to biomarkers and methods for determining sensitivity to EGFR inhibitor, erlotinib HCl. US2004/0209290, Cobleigh et al., concerns gene expression markers for breast cancer prognosis.

Patients to be treated with a HER2 dimerization inhibitor (like pertuzumab as described herein above in more detail) can be selected for therapy based on HER activation or dimerization. Patent publications concerning pertuzumab and selection of patients for therapy therewith include: WO01/00245 (Adams et al.); US2003/0086924 (Sliwkowski, M.); US2004/0013667A1 (Sliwkowski, M.); as well as WO2004/008099A2, and US2004/0106161 (Bossenmaier et al.).

Herceptin™/Trastuzumab is indicated in the art for the treatment of patients with metastatic breast cancer whose tumors overexpress HER2 protein or have HER 2 gene amplification:

a) As monotherapy for the treatment of those patients who have received at least two chemotherapy regimens for their metastatic disease. Prior chemotherapy must have included at least an anthracycline and a taxane unless patients are unsuitable for these treatments. Hormone receptor positive patients must also have received hormonal therapy, unless patients are unsuitable for these treatments,

b) In combination with paclitaxel for the treatment of those patients who have not received chemotherapy for their metastatic disease and for whom an anthracycline is not suitable and

c) In combination with docetaxel for the treatment of those patients who have not received chemotherapy for their metastatic disease.

Herceptin™/Trastuzumab can also be used as adjuvant treatment in early breast cancer. Herceptin™/Trastuzumab is also approved for the treatment of patients with HER2-positive early breast cancer following surgery, chemotherapy (neoadjuvant (i.e. before surgery) or adjuvant), and radiotherapy (if applicable). In addition, Herceptin in combination with capecitabine or 5-fluorouracil and cisplatin is indicated for the treatment of patients with HER2 positive locally advance or metastatic adenocarcinoma of the stomach or gastroesophageal junction who have not received prior anti-cancer treatment for their metastatic disease. The efficacy and safety of neoadjuvant pertuzumab and trastuzumab therapy has been assessed in a phase 2 trial (NEOSPHERE); Gianni (2012) Lancet Oncol 13, 25-32.

In the art, the treatment of breast cancer patients with Herceptin™/Trastuzumab is, for example, recommended and routine for patients having HER2-positive cancer. HER2-positive cancer is present if a high HER2 (protein) expression level detected by immunohistochemical methods (e.g. HER2 (+++)) or HER2 gene amplification detected by in-situ-hybridization (e.g. ISH positive, like a HER2 gene copy number higher than 4 copies of the HER2 gene per tumor cell or ratio of ≥2.0 for the number of HER2 gene copies to the number of signals for CEP17.) or both is found in samples obtained from the patients such as breast tissue biopsies or breast tissue resections or in tissue derived from metastatic sites.

WO 2011/109789, WO 2011/066342, WO 2009/089149 and WO2006/133396 disclose the therapeutic use of PD-L1 inhibitors. Moreover, WO 2010/077634 discloses anti-PD-L1 antibodies and their therapeutic use.

The present invention relates to a method of determining the need of a cancer patient for a PD-L1 inhibitor cotherapy, (i) wherein therapy comprising a modulator of the HER2/neu (ErbB2) signaling pathway and a chemotherapeutic agent is contemplated for the patient or (ii) wherein the patient is undergoing therapy comprising a modulator of the HER2/neu (ErbB2) signaling pathway and a chemotherapeutic agent, said method comprising the steps of
a) measuring in vitro in a sample from said patient the expression level of Estrogen receptor (ER) and of programmed death ligand 1 (PD-L1),
b) determining a patient as being in need of a PD-L1 inhibitor cotherapy if a low or absent ER expression level and an expression level of programmed death ligand 1 (PD-L1) that is increased in comparison to a control is measured in step (a).

Accordingly, the present invention provides a method for determining a cancer patient's need for PD-L1 modulator cotherapy in combination with a modulator of the HER2/neu (ErbB2) signaling pathway and a chemotherapeutic agent, the method comprising the steps of
testing a tumor sample of a patient for whom therapy comprising a modulator of the HER2/neu (ErbB2) signaling pathway and a chemotherapeutic agent is contemplated or who is undergoing said therapy;
determining the expression level of Estrogen receptor (ER) and of programmed death ligand 1 (PD-L1) in said tumor sample,
whereby a low or absent ER expression level and an expression level of programmed death ligand 1 (PD-L1) that is increased in comparison to the control is indicative of a successful use of PD-L1 modulator cotherapy in said patient.

As demonstrated in the appended example, it has been surprisingly found in this invention that Estrogen receptor (ER) negative (ER(−)) cancer patients (cancer patients with a low or even absent ER expression level) undergoing therapy with a modulator of the HER2/neu (ErbB2) signaling pathway (like Herceptin™/Trastuzumab) and a chemotherapeutic agent (like dodetaxel/Taxotere®) show a significantly worse pathological complete response (pCR) to the therapy compared to Estrogen receptor (ER) positive (ER(+)) cancer patients, if the expression level of programmed death ligand 1 (PD-L1) is increased in a sample of the ER negative (ER(−)) cancer patients as compared to a control. The terms “programmed death ligand 1”, “CD274” and “PD-L1” are used interchangeably herein. The ER negative (ER(−)) cancer patients with increased expression level of programmed death ligand 1 (PD-L1) as compared to a control will therefore benefit from additional cotherapy with a PD-L1 inhibitor. It is expected that the pathological complete response rate (pCR) in this patient group will increase, if these patients receive cotherapy with a PD-L1 inhibitor in addition to therapy with a modulator of the HER2/neu (ErbB2) signaling pathway (like Herceptin™/Trastuzumab) and a chemotherapeutic agent (like dodetaxel/Taxotere®). In other words, the ER negative (ER(−)) cancer patients are to receive a programmed death ligand 1 (PD-L1) inhibitor in addition to a modulator of the HER2/neu (ErbB2) signaling pathway (like Trastuzumab) and a chemotherapeutic agent (like dodetaxel/Taxotere®), if the expression level of programmed death ligand 1 (PD-L1) is increased in a sample from the patient in comparison to a control. In the following, ER negative cancer patients or (biological/tumor) samples derived from ER negative cancer patients are denoted herein as “ER(−)”. Likewise ER positive cancer patients or (biological/tumor) samples derived from ER positive cancer patients are denoted herein as “ER(+)”.

In accordance with the above, the present invention relates to a method of treating a cancer in a cancer patient for whom therapy comprising a modulator of the HER2/neu (ErbB2) signaling pathway and a chemotherapeutic agent is contemplated, the method comprising selecting a cancer patient whose cancer is determined to have a low or absent ER expression level and to have an increased expression level of programmed death ligand 1 (PD-L1) in comparison to a control, and administering to the patient an effective amount of a modulator of the HER2/neu (ErbB2) signaling pathway, of a chemotherapeutic agent and of a programmed death ligand 1 (PD-L1) inhibitor. Likewise, the present invention relates to a method of treating a cancer in a cancer patient who is undergoing therapy comprising a modulator of the HER2/neu (ErbB2) signaling pathway and a chemotherapeutic agent, the method comprising selecting a cancer patient whose cancer is determined to have a low or absent ER expression level and to have an increased expression level of programmed death ligand 1 (PD-L1) in comparison to a control, and administering to the patient an effective amount of a programmed death ligand 1 (PD-L1) inhibitor. Herein contemplated is, accordingly, a pharmaceutical composition comprising a modulator of the HER2/neu (ErbB2) signaling pathway, and an inhibitor of programmed death ligand 1 (PD-L1) for use in the treatment of cancer, whereby said cancer is determined to have a low or absent ER expression level and to have an increased expression level of programmed death ligand 1 (PD-L1) in comparison to a control.

In accordance with the above, the herein provided method for determining the need of a cancer patient for a PD-L1 inhibitor cotherapy, may comprise an additional step prior to step a), wherein said step is or comprises obtaining a sample from said cancer patient. Accordingly, the present invention provides a method of determining the need of a cancer patient for a PD-L1 inhibitor cotherapy, (i) wherein therapy comprising a modulator of the HER2/neu (ErbB2) signaling pathway and a chemotherapeutic agent is contemplated for the patient or (ii) wherein the patient is undergoing therapy comprising a modulator of the HER2/neu (ErbB2) signaling pathway and a chemotherapeutic agent, said method comprising a step of obtaining a sample from said cancer patient, the method further comprising the steps

a) measuring in vitro in a sample from said patient the expression level of Estrogen receptor (ER) and of programmed death ligand 1 (PD-L1),

b) determining a patient as being in need of a PD-L1 inhibitor cotherapy if a low or absent ER expression level and an expression level of programmed death ligand 1 (PD-L1) that is increased in comparison to a control is measured in step (a).

Furthermore, it has been found herein and is demonstrated in the appended example, that a patient's need of PD-L1 inhibitor cotherapy can be determined even more reliably, if the expression level of interferon-gamma (IFNγ) is measured in the sample of the patient in addition to the expression level of programmed death ligand 1 (PD-L1). It is shown herein that patients with low or absent ER expression have a significantly worse pathologic complete response to therapy with a modulator of the HER2/neu (ErbB2) signaling pathway and a chemotherapeutic agent, if the expression level of programmed death ligand 1 (PD-L1) is increased and if the expression level of interferon-gamma (IFNγ) is decreased.

Accordingly, the methods provided herein preferably further comprise measuring the expression level of interferon-gamma (IFNγ) in the sample from the patient, whereby a patient is determined to be in need of a PD-L1 inhibitor cotherapy, if the expression level of interferon-gamma (IFNγ) is decreased in comparison to a control. In accordance with the above, the present invention relates in a preferred aspect to a method of determining the need of a cancer patient for a PD-L1 inhibitor cotherapy, (i) wherein therapy comprising a modulator of the HER2/neu (ErbB2) signaling pathway and a chemotherapeutic agent is contemplated for the patient or (ii) wherein the patient is undergoing therapy comprising a modulator of the HER2/neu (ErbB2) signaling pathway and a chemotherapeutic agent, said method comprising the steps of
a) measuring in vitro in a sample from said patient the expression level of Estrogen receptor (ER), the expression level of programmed death ligand 1 (PD-L1), and the expression level of interferon-gamma (IFNγ)
b) determining a patient as being in need of a PD-L1 inhibitor cotherapy if a low or absent ER expression level, an expression level of programmed death ligand 1 (PD-L1) that is increased in comparison to a control, and an expression level of interferon-gamma (IFNγ) that is decreased in comparison to a control is measured in step (a).

Accordingly, an expression level of interferon-gamma (IFNγ) that is decreased in comparison to a control is indicative of a successful use of PD-L1 inhibitor cotherapy in said patient. The herein provided pharmaceutical composition is, in accordance with the above, for use in the treatment of cancer, whereby said cancer is determined to have a low or absent ER expression level, the cancer is determined to have an increased expression level of programmed death ligand 1 (PD-L1) in comparison to a control and the cancer is determined to have a decreased expression level of interferon-gamma (IFNγ) in comparison to the control. Accordingly, a pharmaceutical composition is provided herein comprising a modulator of the HER2/neu (ErbB2) signaling pathway, and an inhibitor of programmed death ligand 1 (PD-L1) for use in the treatment of cancer, whereby said cancer is determined to have a low or absent ER expression level and to have an increased expression level of programmed death ligand 1 (PD-L1) in comparison to a control and to have a decreased expression level of interferon-gamma (IFNγ) in comparison to the control.

The term “cancer patient” as used herein refers to a patient that is suspected to suffer from cancer, suffering from cancer or being prone to suffer from cancer. The cancer to be treated in accordance with the present invention can be a solid cancer, such as breast cancer or gastric cancer. Further, the cancer may be ovarian cancer or colorectal cancer. The cancer is preferably a “HER2-positive” cancer.

Preferably, the cancer is breast cancer, like early breast cancer. The breast cancer may be early stage breast cancer or metastatic breast cancer. Accordingly, the cancer patient (to be treated) is suspected to suffer from solid cancer, is suffering from solid cancer or is being prone to suffer from solid cancer, whereby the solid cancer can be breast cancer or gastric cancer. Preferably, the cancer is breast cancer, like early stage breast cancer. The patient is preferably a human.

As mentioned above, the expression level of Estrogen receptor (ER) and of programmed death ligand (PD-L1), and optionally of interferon-gamma (IFN-γ) can be measured in vitro in a sample from the patient. Preferably, the herein provided methods comprise measuring of interferon-gamma (IFN-γ) in vitro in a sample from the patient. Preferably, the sample to be assessed/analyzed herein is a tumor tissue sample. A patient (or a patient group) is determined as being in need of a PD-L1 inhibitor cotherapy if a low or absent ER expression level and an expression level of programmed death ligand 1 (PD-L1) that is increased in comparison to a control and, optionally, an expression level of interferon-gamma (IFNγ) that is decreased in comparison to the control, is measured in vitro in said sample.

The term “ER” is an abbreviation of “Estrogen receptor”. Likewise, the terms “PD-L1” and “IFN-γ” are abbreviations of the terms “programmed death ligand” and “interferon-gamma”, respectively. Accordingly, the term “ER” can be used interchangeably herein with “Estrogen receptor”. Likewise, the terms “PD-L1” and “IFN-γ” can be used interchangeably herein with the terms “programmed death ligand” and “interferon-gamma”, respectively.

Preferably, the (tumor/biological) sample of the patient and/or the cancer to be treated is characterized by or associated with a low or absent estrogen receptor (ER) expression level. Preferably, the sample of the patient is a tumor sample. The ER expression level can be ER negative (ER(−)). The term “ER(−)” can be used herein interchangeably with the term “ER negative”.

“ER negative” expression level can be determined by routine and standard procedures as described, for example, in the Guideline on Hormone Receptor Testing in Breast Cancer S. Nofech-Mozes, E. Vella, S. Dhesy-Thind, and W. Hanna (A Quality Initiative of the Program in Evidence-Based Care (PEBC), Cancer Care Ontario (CCO); Report Date: Apr. 8, 2011). The Guidelines (and references cited therein) are incorporated by reference in its entirety herein. These Guidelines are available at world wide web at
cancercare.on.ca) and

PEBC Pathology & Laboratory Medicine page at:
cancercare.on.ca/toolbox/qualityguidelines/clin-program/pathlabebs/

Routine and standard procedures for determining the “ER negative” expression level are described in these Guideline and also in the following references:
Nofech-Mozes S, Vella E T, Dhesy-Thind S, Hagerty K L, Mangu P B, Temin S, et al. Systematic review on hormone receptor testing in breast cancer. Applied Immunohistochem Mol Morphol. 2012 May; 20(3):214-63. doi: 10.1097/PAI.0b013e318234aa12. Epub 2011 Nov. 11.
Nofech-Mozes S, Vella E T, Dhesy-Thind S, Hanna W M. Cancer Care Ontario guideline recommendations for hormone receptor testing in breast cancer. Clin Oncol (R Coll Radiol). Epub 2012 May 17.

“ER negative” expression may be determined by IHC (immunohistochemistry), if, for example the expression level of ER is low or absent and/or if the progesterone receptor (PR) expression level is low or absent. The abbreviation “PR” is used herein interchangeably with the term “progesterone receptor”. A sample or patients may be assessed as “ER negative” herein according to the following staining pattern (by IHC):

Only nuclear (not cytoplasmic) staining should be scored.

There are three categories for staining:

Positive: ≥10% staining for ER or PR

Low positive: 1% to 9% staining for ER or PR

Negative: <1% staining for ER and PR

Accordingly, a sample or patients may particularly be assessed as “ER negative” herein if the sample shows the following staining pattern by IHC: <1% staining for ER and PR.

Samples or patients may be assessed as “ER positive” herein if the sample shows a “positive” staining by IHC: ≥1% staining for ER or PR (i.e. more than 1% of the cells examined/assessed have estrogen receptors or progesterone receptors/show staining for estrogen receptors by IHC (immunohistochemistry).

Preferably, a sample or patient is assessed as “ER negative” herein if the sample shows the following staining pattern by IHC::<1% staining for ER (i.e. less than 1% of the cells examined/assessed have estrogen receptors/show staining for estrogen receptor(s) by IHC (immunohistochemistry). Most preferably, a sample or patients is/are assessed as “ER negative” if the nuclei in a tumor tissue sample show <1% staining for ER staining by IHC. Accordingly, from the three categories provided herein above, the assessment of “ER negative” is based on <1% staining for ER by IHC.

Likewise, “ER negative” expression can be determined by further methods routinely employed in the art. For example, “ER negative” may be determined if the mRNA/RNA expression level is low or absent. Routine methods to be used comprise, but are not limited to: Allred score, IRS, Remmele score or any other suitable biochemical detection method. A person skilled in the art is aware that the cut-off for such methods has to match the cut-off as defined above via IHC.

Nucleic acid sequences and amino acid sequences of Progesterone receptor (PR), Estrogen receptor (ER), of programmed death ligand 1 (PD-L1), and/or of interferon-gamma (IFNγ) to be used herein are well known and can be retrieved from databases like NCBI. Exemplary sequences are provided herein (see for example SEQ ID NO: 38-51).

The methods and sample types used for establishing a cut-off value of a marker (like programmed death ligand 1 (PD-L1) and/or interferon-gamma (IFN-γ)) and for measuring the sample obtained from an individual or patient to be analyzed match each other or are the same. Cut-off values, i.e. values above which overexpression (e.g. increased expression of programmed death ligand 1 (PD-L1) in comparison to a control) is acknowledged can be obtained in a control group. Cut-off values, i.e. values below which decreased expression (e.g. decreased expression of interferon-gamma (IFN-γ) in comparison to a control) is acknowledged can be obtained in a control group.

The control group on which the cut-off value is based is chosen to match the group of individuals/patients under investigation. In other words, if the method of the present invention is used to determine the need for PD-L1 cotherapy in patients with breast cancer or gastric cancer, respectively, the control group is also patients with breast cancer or gastric cancer, respectively. The control group used to establish the cut-off values for both, PDL-1 and IFN-γ, respectively), comprises at least 40, or at least 50, or at least 100 individuals/patients. An expression level or corresponding value above the cut-off is considered to represent overexpression and a value at or below the cut-off is considered as decreased expression.

In one embodiment, the “IFN-γ” expression level in a tumor tissue sample from an individual/patient is compared to a cut-off value. A value above the cut-off is considered to represent overexpression of IFN-γ and a value at or below the cut-off is considered as decreased expression of IFN-γ. In one embodiment the decreased expression is acknowledged if the expression level for IFN-γ is at or below the value of the highest quintile, quartile or tertile, respectively, as established in the control group. In one embodiment the cut-off for IFN-γ is the highest tertile. In one embodiment the cut-off value is a value between the 70^{th} and the 80^{th} percentile. In one embodiment the cut-off value for IFN-γ is the 73^{rd} percentile, i.e a value above this cut-off is considered to represent overexpression of IFN-γ and a value at or below the 73^{rd} percentile is considered as decreased expression of IFN-γ. In one embodiment, individuals/patients are determined as being in need of a PD-L1 cotherapy, if IFN-γ expression in a sample (like a tumor tissue sample) is decreased (i.e. below or at the IFN-γ cut-off value) In one embodiment individuals/patients are determined as not being in need of a PDL-1 cotherapy, if IFN-γ is overexpressed (i.e. above the IFN-γ cut-off value as described above).

In one embodiment the PD-L1 expression level, in a tumor tissue sample from an individual/patient is compared to a cut-off value. A value above the cut-off is considered to represent overexpression of PD-L1 and a value at or below the cut-off is considered as decreased expression of PD-L1. In one embodiment overexpression for PDL-1 is acknowledged if the expression level for PDL-1 is above a cut-off value between the 50^{th} percentile and the 75^{th} percentile, as established in a control group. In one embodiment overexpression for PDL-1 is acknowledged if the expression level for PDL-1 is above a cut-off value between the 50^{th} percentile and the 70^{th} percentile, of the control group. In one embodiment individuals/patients are determined as being in need of a PDL-1 cotherapy, if PDL-1 is overexpressed (i.e. the PDL-1 expression level determined is above the PDL-1 cut-off value).

In one further embodiment overexpression for PDL-1 is established in the sub-group of individuals/patients having a decreased expression level of IFN-γ in a tumor tissue sample. In one embodiment overexpression for PDL-1 is acknowledged if the expression level for PDL-1 is above a cut-off value between the 40^{th} percentile and the 65^{th} percentile, as established in this sub-group. In one embodiment overexpression for PDL-1 is acknowledged if the expression level for PDL-1 is above a cut-off value between the 50^{th} percentile and the 60^{th} percentile, as established in this sub-group. In one embodiment individuals/patients are determined as being in need of a PDL-1 cotherapy, if the PDL-1 expression level in the sub-group with decreased expression of IFN-γ is above the 54^{th} percentile.

In one embodiment, individuals/patients are determined as being in need of a PDL-1 cotherapy, if IFN-γ expression in a tumor tissue sample is decreased (i.e. below or at the IFN-γ cut-off value) and PDL-1 is overexpressed (i.e. above the PDL-1 cut-off value).

The term “expression level of programmed death ligand 1 (PD-L1) that is increased in comparison to a control” can be used interchangeably herein with “expression level of programmed death ligand 1 (PD-L1) above the PDL-1 cut-off value” as defined and explained herein above.

The term “expression level of interferon-gamma (IFNγ) that is decreased in comparison to a control” can be used interchangeably herein with “expression level of interferon-gamma (IFNγ) below or at the IFNγ cut-off value”.

The present invention relates to the following aspects.

The present invention relates to a method of determining the need of a cancer patient for a PD-L1 inhibitor cotherapy, (i) wherein therapy comprising a modulator of the HER2/neu (ErbB2) signaling pathway and a chemotherapeutic agent is contemplated for the patient or (ii) wherein the patient is undergoing therapy comprising a modulator of the HER2/neu (ErbB2) signaling pathway and a chemotherapeutic agent, the method comprising the steps of
a) measuring in vitro in a sample from said patient the expression level of Estrogen receptor (ER), of programmed death ligand 1 (PD-L1), and of interferon-gamma (IFNγ);
b) determining a patient as being in need of a PD-L1 inhibitor cotherapy if a low or absent ER expression level (like ER(−)/ER-negative), an expression level of programmed death ligand 1 (PD-L1) above the PDL-1 cut-off value and an expression level of interferon-gamma (IFNγ) below or at the IFNγ cut-off value is measured in step (a).

The present invention relates to a method of treating a cancer in a cancer patient for whom therapy comprising a modulator of the HER2/neu (ErbB2) signaling pathway and a chemotherapeutic agent is contemplated, the method comprising selecting a cancer patient whose cancer is determined to have a low or absent ER expression level (like ER(−)/ER-negative) and to have an expression level of programmed death ligand 1 (PD-L1) above the PDL-1 cut-off value and to have an expression level of interferon-gamma (IFNγ) below or at the IFNγ cut-off value, and administering to the patient an effective amount of a modulator of the HER2/neu (ErbB2) signaling pathway, of a chemotherapeutic agent and of a programmed death ligand 1 (PD-L1) inhibitor.

The present invention relates to a method of treating a cancer in a cancer patient who is undergoing therapy comprising a modulator of the HER2/neu (ErbB2) signaling pathway and a chemotherapeutic agent, the method comprising selecting a cancer patient whose cancer is determined to have a low or absent ER expression level (like ER(−)/ER-negative) and to have an expression level of programmed death ligand 1 (PD-L1) above the PDL-1 cut-off value and to have an expression level of interferon-gamma (IFNγ) below or at the IFNγ cut-off value, and administering to the patient an effective amount of a programmed death ligand 1 (PD-L1) inhibitor.

The present invention relates to a pharmaceutical composition comprising a modulator of the HER2/neu (ErbB2) signaling pathway, and an inhibitor of programmed death ligand 1 (PD-L1) for use in the treatment of cancer, whereby said cancer is determined to have a low or absent ER expression level (like ER(−)/ER-negative) and to have an expression level of programmed death ligand 1 (PD-L1) above the PDL-1 cut-off value and to have an expression level of interferon-gamma (IFNγ) below or at the IFNγ cut-off value.

All explanations and definitions given herein for “PD-L1 inhibitor”, “PD-L1 inhibitor cotherapy”, “cancer”, “cancer patient”, “modulator of the HER2/neu (ErbB2) signaling pathway”, “chemotherapeutic agent”, “sample”, “expression level” and the like apply, mutatis mutandis, to the above aspects of the present invention.

The expression level of Estrogen receptor (ER), of programmed death ligand 1 (PD-L1), and of interferon-gamma (IFNγ) in a sample from the patient may be measured in vitro simultaneously or subsequently in any combination. For example, the expression level of Estrogen receptor (ER), of programmed death ligand 1 (PD-L1), and of interferon-gamma (IFNγ) may be measured simultaneously. The expression level of Estrogen receptor (ER) may be measured first, followed by the measurement of programmed death ligand 1 (PD-L1) and of interferon-gamma (IFNγ). The expression level of programmed death ligand 1 (PD-L1) may be measured first, followed by the (simultaneous or subsequent) measurement of Estrogen receptor (ER) and of interferon-gamma (IFNγ). The expression level of interferon-gamma (IFNγ) may be measured first, followed by the (simultaneous or subsequent) measurement of Estrogen receptor (ER) and of programmed death ligand 1 (PD-L1). Any order/combination of the measurement of the expression level of Estrogen receptor (ER), of programmed death ligand 1 (PD-L1), and of interferon-gamma (IFNγ) in a sample from the patient is envisaged and comprised herein.

Herein contemplated is a determination of a patient as being in need of a PD-L1 inhibitor cotherapy if, in a first step (1) a low or absent ER expression level (like ER(−)/ER-negative) is measured, and if, in a second step (2) an expression level of interferon-gamma (IFNγ) below or at the IFNγ cut-off value is measured and if, in a third step (3) an expression level of programmed death ligand 1 (PD-L1) above the PDL-1 cut-off value is measured.

The present invention relates to the following aspects: The present invention relates to a method of determining the need of a cancer patient for a PD-L1 inhibitor cotherapy, (i) wherein therapy comprising a modulator of the HER2/neu (ErbB2) signaling pathway and a chemotherapeutic agent is contemplated for the patient or (ii) wherein the patient is undergoing therapy comprising a modulator of the HER2/neu (ErbB2) signaling pathway and a chemotherapeutic agent, the method comprising the steps of
a) measuring in vitro in a sample from said patient the expression level of Estrogen receptor (ER), of programmed death ligand 1 (PD-L1), and of interferon-gamma (IFNγ);
b) determining a patient as being in need of a PD-L1 inhibitor cotherapy if, in a first step (1) a low or absent ER expression level (like ER(−)/ER-negative) is measured, and if in a second step (2) an expression level of interferon-gamma (IFNγ) below or at the IFNγ cut-off value is measured and if in a third step (3) an expression level of programmed death ligand 1 (PD-L1) above the PDL-1 cut-off value is measured.

The present invention relates to a method of treating a cancer in a cancer patient for whom therapy comprising a modulator of the HER2/neu (ErbB2) signaling pathway and a chemotherapeutic agent is contemplated, the method comprising selecting a cancer patient whose cancer is determined to have in a first step (1) a low or absent ER expression level (like ER(−)/ER-negative) and in a second step (2) to have an expression level of interferon-gamma (IFNγ) below or at the IFNγ cut-off value, and in a third step (3) to have an expression level of programmed death ligand 1 (PD-L1) above the PDL-1 cut-off value, and administering to the patient an effective amount of a modulator of the HER2/neu (ErbB2) signaling pathway, of a chemotherapeutic agent and of a programmed death ligand 1 (PD-L1) inhibitor.

The present invention relates to a method of treating a cancer in a cancer patient who is undergoing therapy comprising a modulator of the HER2/neu (ErbB2) signaling pathway and a chemotherapeutic agent, the method comprising selecting a cancer patient whose cancer is determined to have in a first step (1) a low or absent ER expression level (like ER(−)/ER-negative) and in a second step (2) to have an expression level of interferon-gamma (IFNγ) below or at the IFNγ cut-off value, and in a third step (3) to have an expression level of programmed death ligand 1 (PD-L1) above the PDL-1 cut-off value, and administering to the patient an effective amount of a programmed death ligand 1 (PD-L1) inhibitor.

The present invention relates to a pharmaceutical composition comprising a modulator of the HER2/neu (ErbB2) signaling pathway, and an inhibitor of programmed death ligand 1 (PD-L1) for use in the treatment of cancer, whereby said cancer is determined to have a low or absent ER expression level (like ER(−)/ER-negative), to have an expression level of interferon-gamma (IFNγ) below or at the IFNγ cut-off value, to have an expression level of programmed death ligand 1 (PD-L1) above the PDL-1 cut-off value.

All explanations and definitions given herein for “PD-L1 inhibitor”, “PD-L1 inhibitor cotherapy”, “cancer”, “cancer patient”, “modulator of the HER2/neu (ErbB2) signaling pathway”, “chemotherapeutic agent”, “sample”, “expression level” and the like apply, mutatis mutandis, to the above aspects of the present invention.

The following relates to an exemplary cut-off value allowing determining a patient as being in need of a PD-L1 inhibitor cotherapy in accordance with the present invention. It can be easily determined by routine techniques (such as Affymetrix) whether the expression level of PD-L1 and/or IFN-gamma in a sample from a patient is below or above such cut-off values.

If a gene expression analysis gives a result for IFN-gamma expression higher or equal to 4.8 no combination treatment (HER2-targeted and PDL1-targeted) is recommended and no further PDL1 assessment is necessary. If a gene expression analysis gives a result for IFN-gamma lower than 4.8 a parallel assessment of PDL-1 is necessary. If PDL-1 gene expression analysis then gives a result of higher or equal to 5.3 a combination treatment (HER2-targeted and PDL1-targeted) is recommended. This exemplary protocol is illustrated in FIG. 19.

In this context Affymetrix can be performed as follows: Total RNA from tumor cells was extracted FFPE tumor sections using Light Cycler Pertuzumab FFPET RNA Kit (Roche Diagnostics). RNA was processed for hybridization using the WT-Ovation FFPE System V2 (Nugen) and hybridized to Affymetrix GeneChip® Human Genome U133 Plus 2.0 Arrays. Hybridized arrays were washed and stained on Affymetrix Fluidics Station 450 and scanned with an Affymetrix GeneChip® Scanner 3000 7G.

As mentioned the expression level of PD-L1 and/or IFN-gamma in a sample from a patient can be determined by routine techniques, such as Affymetrix. The following relates an exemplary protocol for such a determination (also termed herein Gene Expression Profiling):

The tumor biopsy samples can be profiled for gene expression on AFFYMETRIX HG-U133Plus 2 whole Human Genome microarray platform. Roche HighPure RNA extraction, NuGen amplification and standard AFFYMETRIX hybridization and scanning protocols can be used. These protocols etc. are incorporated herein by reference. All array scans usually pass standard AFFYMETRIX QC. Robust Multiarray algorithm (RMA) can be used for preprocessing of raw signals (Irizarry et al, 2003. World wide web at ncbi.nlm.nih.gov/pubmed/12925520; incorporated herein by reference). All probe sets available for the genes of interest can be retrieved as reported below. For gene CD274, when several probe sets were available to represent this gene, the probe set with the highest average expression value (defined as an arithmetical average of expression of a given probe set) was selected to represent the gene:

CD274 (PDL1)

223834_at selected for PDL1

227458_at

The selected probe set corresponds to the last exon/3′UTR of the gene and captures all known RefSEq mRNAs (see FIG. 6)

IFNG

210354_at

This probe set also represents the last exon/3′UTR of the gene and captures all known RefSEq mRNAs (see FIG. 7)

In accordance with the above, the expression level of Interferon-gamma may be measured prior to the expression level of Estrogen receptor (ER) and/or prior to the expression level of programmed death ligand 1 (PD-L1). The step of measuring the expression level of Estrogen receptor (ER) and of programmed death ligand 1 (PD-L1) may even be absent.

As shown in the appended Example, PD-L1 cotherapy can, for example, not be recommended if the expression level of interferon-gamma (IFNγ) is higher or equal to (about) 4.8 as determined by routine methods like Affymetrix.

Accordingly, the present invention provides a method of determining the need of a cancer patient for a PD-L1 inhibitor cotherapy, wherein therapy comprising a modulator of the HER2/neu (ErbB2) signaling pathway and a chemotherapeutic agent is contemplated for the patient or wherein the patient is undergoing therapy comprising a modulator of the HER2/neu (ErbB2) signaling pathway and a chemotherapeutic agent, the method comprising the steps

(a) measuring in vitro in a sample from said patient the expression level of interferon-gamma (IFNγ)

(b) determining a patient as being not in need of a PD-L1 inhibitor cotherapy if the expression level of interferon-gamma (IFNγ) is higher or equal to (about) 4.8 as determined by routine methods like Affymetrix in step (a).

If the expression level of interferon-gamma (IFNγ) is lower than (about) 4.8 as determined by routine methods like Affymetrix, the expression level of programmed death ligand 1 (PD-L1) and, optionally, Estrogen receptor (ER) can be measured in vitro in a sample from said patient.

Accordingly, the present invention provides a method of determining the need of a cancer patient for a PD-L1 inhibitor cotherapy, wherein therapy comprising a modulator of the HER2/neu (ErbB2) signaling pathway and a chemotherapeutic agent is contemplated for the patient or wherein the patient is undergoing therapy comprising a modulator of the HER2/neu (ErbB2) signaling pathway and a chemotherapeutic agent, the method comprising the steps

(a) measuring in vitro in a sample from said patient the expression level of interferon-gamma (IFNγ), Estrogen receptor (ER) and of programmed death ligand 1 (PD-L1),

(b) determining a patient as being in need of a PD-L1 inhibitor cotherapy if the expression level of interferon-gamma (IFNγ) is lower than (about) 4.8 as determined by routine methods like Affymetrix, and if a low or absent ER expression level and, optionally, an expression level of programmed death ligand 1 (PD-L1) that is increased in comparison to a control is measured in step (a).

A patient can be determined in accordance with the present invention to be in need of PD-L1 inhibitor cotherapy if the expression level of programmed death ligand 1 (PD-L1) measured in the sample from the patient is increased in comparison to a control. For example, the expression level of programmed death ligand 1 (PD-L1) can be higher or equal to (about) 5.3 determined by routine methods like Affymetrix.

All explanations and definitions given herein for “PD-L1 inhibitor”, “PD-L1 inhibitor cotherapy”, “cancer”, “cancer patient”, “modulator of the HER2/neu (ErbB2) signaling pathway”, “chemotherapeutic agent”, “sample”, “expression level” and the like as given herein apply, mutatis mutandis, in this context.

Accordingly, the present invention relates to a method of treating a cancer in a cancer patient for whom therapy comprising a modulator of the HER2/neu (ErbB2) signaling pathway and a chemotherapeutic agent is contemplated, the method comprising selecting a cancer patient whose cancer is determined to have a low or absent ER expression level and to have an increased expression level of programmed death ligand 1 (PD-L1) in comparison to a control, and an expression level of interferon-gamma (IFNγ) that is lower than (about) 4.8 as determined by routine methods like Affymetrix, and administering to the patient an effective amount of a modulator of the HER2/neu (ErbB2) signaling pathway, of a chemotherapeutic agent and of a programmed death ligand 1 (PD-L1) inhibitor.

Furthermore, the present invention relates to a method of treating a cancer in a cancer patient who is undergoing therapy comprising a modulator of the HER2/neu (ErbB2) signaling pathway and a chemotherapeutic agent, the method comprising selecting a cancer patient whose cancer is determined to have a low or absent ER expression level and to have an increased expression level of programmed death ligand 1 (PD-L1) in comparison to a control, and to have an expression level of interferon-gamma (IFNγ) that is lower than (about) 4.8 as determined by routine methods like Affymetrix, and administering to the patient an effective amount of a programmed death ligand 1 (PD-L1) inhibitor.

A pharmaceutical composition is provided comprising a modulator of the HER2/neu (ErbB2) signaling pathway, and an inhibitor of programmed death ligand 1 (PD-L1) for use in the treatment of cancer, whereby said cancer is determined to have a low or absent ER expression level and to have an increased expression level of programmed death ligand 1 (PD-L1) in comparison to a control, and an expression level of interferon-gamma (IFNγ) that is lower than (about) 4.8 as determined by routine methods like Affymetrix.

The pharmaceutical composition for use in the treatment of cancer may further comprise a chemotherapeutic agent.

In accordance with the above, the herein provided methods may comprise a step of measuring the expression level of Interferon-gamma (IFNγ) in said sample and determining a patient as being in need of a PD-L1 inhibitor cotherapy if an expression level of interferon-gamma (IFNγ) that is decreased in comparison to the control is measured. For example, a “decreased expression level” of interferon-gamma (IFNγ) may be an expression level lower than (about) 4.8 as determined by routine methods like Affymetrix. Accordingly, the cancer that is determined to have a decreased expression level of interferon-gamma (IFNγ) in comparison to the control may be determined to have an expression level of interferon-gamma (IFNγ) that is lower than (about) 4.8 as determined by routine methods like Affymetrix,

It is envisaged herein that the expression level may be reflected in the activity of the gene product/protein. Accordingly, also the activity of ER, PD-L1 and/or IFN-γ can be measured and evaluated in addition or in the alternative to the expression level in accordance with the present invention. A person skilled in the art is aware of corresponding means and methods for detecting and evaluating the ER, PD-L1 and IFN-γ expression level and/or activity. Exemplary methods to be used include but are not limited to molecular assessments such as Western Blots, Northern Blots, Real-Time PCR and the like. Such methods are described herein in detail.

The expression level of ER, PD-L1 and/or IFN-γ may be the mRNA expression level of ER, PD-L1 and/or IFN-γ. If the gene product is an RNA, in particular an mRNA (e.g. unspliced, partially spliced or spliced mRNA), determination can be performed by taking advantage of northern blotting techniques, in situ hybridization, hybridization on microarrays or DNA chips equipped with one or more probes or probe sets specific for mRNA transcripts or PCR techniques, like, quantitative PCR techniques, such as Real time PCR. These and other suitable methods for binding (specific) mRNA are well known in the art and are, for example, described in Sambrook and Russell (2001, loc. cit.). A skilled person is capable of determining the amount of the component, in particular said gene products, by taking advantage of a correlation, preferably a linear correlation, between the intensity of a detection signal and the amount of the gene product to be determined.

The expression level may be the protein expression level of ER, PD-L1 and/or IFN-γ. Quantification of the protein expression level can be performed by taking advantage of the well known techniques such as western blotting techniques, immunoassays, gel- or blot-based methods, IHC, mass spectrometry, flow cytometry, FACS and the like. Generally, a person skilled in the art is aware of methods for the quantitation of (a) polypeptide(s)/protein(s). Amounts of purified polypeptide in solution can be determined by physical methods, e.g. photometry. Methods of quantifying a particular polypeptide in a mixture may rely on specific binding, e.g of antibodies. Specific detection and quantitation methods exploiting the specificity of antibodies comprise for example immunohistochemistry (in situ). Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

Alternatively, protein quantitation methods may involve but are not limited to mass spectrometry or enzyme-linked immunosorbant assay methods.

Also, the use of high throughput screening (HTS) is envisaged in the context of the present invention. Suitable (HTS) approaches are known in the art. A person skilled in the art is readily in the position to adapt such protocols or known HTS approaches to the performance of the methods of the present invention. Such assays are usually performed in liquid phase, wherein for each cell/tissue/cell culture to be tested at least one reaction batch is made. Typical containers to be used are micro titer plates having for example, 384, 1536, or 3456 wells (i.e. multiples of the “original” 96 reaction vessels). Robotics, data processing and control software, and sensitive detectors, are further commonly used components of a HTS device. Often robot systems are used to transport micro titer plates from station to station for addition and mixing of sample(s) and reagent(s), incubating the reagents and final readout (detection). Usually, HTS can be used in the simultaneous preparation, incubation and analysis of many plates. The assay can be performed in a single reaction (which is usually preferred), may, however, also comprise washing and/or transfer steps. Detection can be performed taking advantage of radioactivity, luminescence or fluorescence, like fluorescence-resonance-energy transfer (FRET) and fluorescence polarisation (FP) and the like. The biological samples described herein can also be used in such a context. In particular, cellular assays and in vivo assays can be employed in HTS. Cellular assays may also comprise cellular extracts, i.e. extracts from cells, tissues and the like. However, preferred herein is the use of cell(s) or tissue(s) as biological sample (in particular a sample obtained from a patient/subject suffering or being prone to suffer from cancer), whereas in vivo assays are particularly useful in the validation of modulators/inhibitors/chemotherapeutic agents to be used herein. Depending on the results of a first assay, follow up assays can be performed by re-running the experiment to collect further data on a narrowed set (e.g. samples found “positive” in the first assay), confirming and refining observations.

As used in context of the methods of the present invention, a non-limiting example of a “control” is preferably a control from a patient who is not in need of a PD-L1 inhibitor cotherapy, for example a sample/cell/tissue obtained from one or more healthy subjects or one or more patients that suffer from a cancer/tumor and are known to be not in need of a PD-L1 inhibitor cotherapy treatment. For example, such a control (sample) may be from a patient who does not benefit from additional PD-L1 inhibitor cotherapy. Another non-limiting example of a “control” is an “internal standard”, for example a mixture of purified or synthetically produced proteins and/or peptides or RNA, where the amounts of each protein/peptide/RNA is gauged by using the control described above.

A further non-limiting example of a “control” may be a “healthy” control, for example a sample/cell/tissue obtained from a healthy subject or patient that is not suffering from a cancer/tumor or a cell obtained from such a subject. In accordance with the above, the reference or control expression level of ER, PD-L1 and/or IFN-γ is that determined in (a sample of) the corresponding healthy control subject/patient, i.e. it is the “normal” status of ER, PD-L1 and/or IFN-γ. The control may also be a sample/cell/tissue obtained from the individual or patient suspected of suffering from the cancer provided that the sample/cell/tissue does not contain tumor or cancer cells. In a further alternative, the “control” may be a sample/cell/tissue obtained from an individual or patient suffering from the cancer, that has been obtained prior to the development or diagnosis of said cancer.

The sample to be assessed in accordance with the herein provided methods may comprise non-diseased cells and/or diseased cells, i.e. non-cancerous cells and/or cancerous cells. However, the content of cancerous cells among non-cancerous cells should be higher than for example 50%. The sample may also (or even solely) comprise cancer/tumor cell(s), such as breast cancer/tumor cell(s). The term “sample” shall generally mean any biological sample obtained from a patient's tumor. The sample may be a tissue resection or a tissue biopsy. The sample may also be a metastatic lesion or a section of a metastatic lesion or a blood sample known or suspected to comprise circulating tumor cells. In accordance with the above, the biological sample may comprise cancer cells and to a certain extent i.e. less than for example 50% non-cancer cells (other cells). The skilled pathologist is able to differentiate cancer cells from normal tissue cells. Methods for obtaining tissue biopsies, tissue resections and body fluids and the like from mammals, such as humans, are well known in the art.

As explained above, the cancer patient who is determined to be in need of PD-L1 inhibitor cotherapy in accordance with the present invention is undergoing therapy comprising a modulator of the HER2/neu (ErbB2) signaling pathway and a chemotherapeutic agent or such a therapy comprising a modulator of the HER2/neu (ErbB2) signaling pathway and a chemotherapeutic agent is contemplated for the patient. Therapy comprising a modulator of the HER2/neu (ErbB2) signaling pathway and a chemotherapeutic agent is indicated for patients with “HER2-positive cancer”, like a patient that is suspected to suffer from a HER2-positive cancer, suffering from a HER2-positive cancer or being prone to suffer from a HER2-positive cancer. Preferably, the cancer to be treated is in accordance with the present invention a “HER2-positive cancer”, particularly a “HER2-positive breast cancer”. A “HER2-positive cancer” can be a “HER2-positive breast cancer” or a “HER2-positive gastric cancer”. Further, the HER2-positive cancer may be ovarian cancer, lung cancer, colorectal cancer, kidney cancer, bone cancer, bone marrow cancer, bladder cancer, skin cancer, prostate cancer, esophagus cancer, salivary gland cancer, pancreas cancer, liver cancer, head and neck cancer, CNS (especially brain) cancer, cervix cancer, cartilage cancer, colon cancer, genitourinary cancer, gastrointestinal tract cancer, pancreas cancer, synovium cancer, testis cancer, thymus cancer, thyroid cancer and uterine cancer.

The term “HER2-positive cancer” as used herein refers to a cancer/tumorous tissue etc. which comprises cancer cells which have higher than normal levels of HER2. For the purpose of the present invention, “HER2-positive cancer” has an immunohistochemistry (IHC) score of at least 2+ and/or an in situ hybridization (ISH) amplification ratio ≥2.0 (i.e. is ISH-positive). Accordingly, HER2-positive cancer is present if a high HER2 (protein) expression level detected e.g. by immunohistochemical methods and/or HER2 gene amplification detected by in-situ-hybridization (ISH positive, like a HER2 gene copy number higher than 4 copies of the HER2 gene per tumor cell or ratio of ≥2.0 for the number of HER2 gene copies to the number of signals for CEP17.) is found in samples obtained from the patients such as breast tissue biopsies or breast tissue resections or in tissue derived from metastatic sites. In one embodiment “HER2-positive cancer” has an immunohistochemistry (IHC) score of HER2(3+) and/or is ISH positive.

The expression level of HER2 may be detected by an immunohistochemical method, whereas said HER2 gene amplification status can be measured with in situ hybridization methods, like fluorescence in situ hybridization techniques (FISH). Corresponding assays and kits are well known in the art, for protein expression assays as well as for the detection of gene amplifications. Alternatively, other methods like qRT-PCR might be used to detect levels of HER2 gene expression.

The expression level of HER2 can, inter alia, be detected by an immunohistochemical method. Such methods are well known in the art and corresponding commercial kits are available. Exemplary kits which may be used in accordance with the present invention are, inter alia, HerceptTest™ produced and distributed by the company Dako or the test called Ventana Pathway™. The level of HER2 protein expression may be assessed by using the reagents provided with and following the protocol of the HercepTest™. A skilled person will be aware of further means and methods for determining the expression level of HER2 by immunohistochemical methods; see for example WO 2005/117553. Therefore, the expression level of HER2 can be easily and reproducibly determined by a person skilled in the art without undue burden. However, to ensure accurate and reproducible results, the testing must be performed in a specialized laboratory, which can ensure validation of the testing procedures. The expression level of HER2 can be classified in a low expression level, an intermediate expression level and a high expression level. It is preferred in context of this invention that HER2-positive disease is defined by a strong expression level of HER2 (e.g. HER2(3+) by IHC), for example determined in a sample of a cancer patient.

The recommended scoring system to evaluate the IHC staining patterns which reflects the expression levels of HER2 designated herein HER2(0), HER2(+), HER2(++) and HER2(+++), is as follows:

| Staining | | HER2 |
|---|---|---|
| Intensity | | overexpression |
| Score | Staining Pattern | assessment |
| 0 | No staining is observed or membrane staining is | negative |
| | observed in <10% of the tumor cells | |
| 1+ | A faint/barely perceptible membrane staining is | negative |
| | detected in >10% of the tumor cells. the cells | |
| | are only stained in part of their membrane. | |
| 2+ | A weak to moderate complete staining is | weak to |
| | detected in >10% of the tumor cells. | moderate |
| | | overexpression. |
| 3+ | A strong complete membrane staining is | strong |
| | detected in >10% of the tumor cells. | overexpression. |

The above IHC staining patterns are routinely used in determining HER2-positive breast cancer. The terms HER2(+), HER2(++) and HER2(+++) used herein are equivalent to the terms HER2(1+), HER2(2+) and HER2(3+). A “low protein expression level” used in context of this invention corresponds to a 0 or 1+ score (“negative assessment” according to the table shown herein above), an “weak to moderate protein expression level” corresponds to a 2+ score (“weak to moderate overexpression”, see the table above) and a “high protein expression level” corresponds to a 3+ score (“strong overexpression”, see the table above). As described herein above in detail, the evaluation of the protein expression level (i.e. the scoring system as shown in the table) is based on results obtained by immunohistochemical methods. As a standard or routinely, the HER-2 status is, accordingly, performed by immunohistochemistry with one of two FDA-approved commercial kits available; namely the Dako Herceptest™ and the Ventana Pathway™. These are semi-quantitative assays which stratify expression levels into 0 (<20,000 receptors per cell, no expression visible by IHC staining), 1+ (˜100,000 receptors per cell, partial membrane staining, <10% of cells overexpressing HER-2), 2+ (˜500,000 receptors per cell, light to moderate complete membrane staining, >10% of cells overexpressing HER-2), and 3+ (˜2,000,000 receptors per cell, strong complete membrane staining, >10% of cells overexpressing HER-2).

Alternatively, further methods for the evaluation of the protein expression level of HER2 may be used, e.g. Western Blots, ELISA-based detection systems and so on.

A HER2-positive cancer may also be diagnosed by assessing the gene amplification status of HER2. HER2-positive cancer is, accordingly, diagnosed if this assessment by ISH is positive. In accordance with this assessment, a HER2-positive cancer may, inter alia, relate to an average HER2 gene copy number higher than 4 copies of the HER2 gene per tumor cell (for those test systems without an internal centromere control probe) or to a HER2/CEP17 ratio of >=2.0 (for those test systems using an internal chromosome 17 centromere control probe). In other words, the HER2-positive cancer may, inter alia, relate to a HER2 gene copy number greater than 4. The amplification level of the HER2 gene may easily be identified by in situ hybridization (ISH) like fluorescent in situ hybridization (FISH), chromogenic in situ hybridization (CISH) and silver in situ hybridization (SISH). These methods are known to the skilled artisan. The principles of these methods can be deduced from standard text books. Commercial kits for the determination of the HER2 gene amplification status by in situ hybridization are available.

The below IHC staining patterns are recommended for determining HER2-positive gastric cancer (see Dako Herceptest package insert).

of Hercep Test™ stained biopsies a cluster of at least 5 stained tumor cells is recommended. A cluster of at least 5 stained tumor cells consists of 5 connected HER2 stained tumor cells.

TABLE 9
Interpretation and scoring of HER2 immunohistochemical staining

| | | | HER2 |
|---|---|---|---|
| | | | Over- |
| | | | expres- |
| | | | sion |
| | Surgical Specimen- | Biopsy Specimen- | Assess- |
| Score | Staining Pattern | Staining Pattern | ment |
| 0 | No reactivity or | No reactivity or no | Negative |
| | membranous reactivity in | membranous reactivity in any | |
| | <10% of tumor cells | (or <5 clustered) tumor cell | |
| 1+ | Faint/barely perceptible | Tumor cell cluster (≥5 cells) | Negative |
| | membranous reactivity | with a faint/barely perceptible | |
| | in ≥10% of tumor cells, | membranous reactivity | |
| | cells are reactive only in | irrespective of percentage | |
| | part of their membrane | of tumor cells stained | |
| 2+ | Weak to moderate | Tumor cell cluster (≥5 cells) | Equivo- |
| | complete, basolateral | with a weak to moderate | cal |
| | or lateral membranous | complete, basolateral | |
| | reactivity in ≥10% | or lateral membranous | |
| | of tumor cells | reactivity irrespective | |
| | | of percentage of tumor | |
| | | cells stained | |
| 3+ | Strong complete, | Tumor cell cluster (≥5 cells) | Positive |
| | basolateral or lateral | with a strong complete, | |
| | membranous reactivity | basolateral or lateral | |
| | in ≥10% of tumor cells | membranous reactivity | |
| | | irrespective of percentage | |
| | | of tumor cells stained | |

Guidelines based on Hofmann et al.(40).

More refined IHC staining patterns for determining HER2-positive gastric cancer is as follows:

| Staining | | | HER2 |
|---|---|---|---|
| Intensity | Surgical specimen- | Biopsy specimen- | Overexpression |
| Score | staining pattern | staining pattern | Assessment |
| 0 | No reactivity or no | No reactivity or no | Negative |
| | membranous reactivity in | membranous reactivity in any | |
| | <10% of tumour cells | tumour cell | |
| 1+ | Faint/barely perceptible | Tumour cell cluster (≥5 cells) | Negative |
| | membranous reactivity in | with a faint/barely perceptible | |
| | ≥10% of tumour cells; cells | membranous reactivity | |
| | are reactive only in part of | irrespective of percentage of | |
| | their membrane | tumour cells stained | |
| 2+ | Weak to moderate complete, | Tumour cell cluster (≥5 cells) | Equivocal |
| | basolateral or lateral | with a weak to moderate | |
| | membranous reactivity in | complete, basolateral or lateral | |
| | ≥10% of tumour cells | membranous reactivity | |
| | | irrespective of percentage of | |
| | | tumour cells stained | |
| 3+ | Strong complete, basolateral | Tumour cell cluster (≥5 cells) | Positive |
| | or lateral membranous | with a strong complete, | |
| | reactivity in ≥10% of | basolateral or lateral | |
| | tumour cells | membranous reactivity | |
| | | irrespective of percentage of | |
| | | tumour cells stained | |

As indicated above, the HER2 positive cancer to be treated in accordance with the present invention may be breast cancer, such early stage breast cancer. The term “early-stage breast cancer” as used herein refers to breast cancer that has not spread beyond the breast or the auxiliary lymph nodes. Such cancer can be generally treated with neoadjuvant or adjuvant therapy. The term “neoadjuvant therapy” as used herein refers to systemic therapy given prior to surgery. The term “adjuvant therapy” refers to systemic therapy given after surgery. In accordance with the above, treatment may be neoadjuvant or adjuvant therapy of early-stage breast cancer.

In accordance with the above, the sample to be assessed can be (obtained) from a patient with HER2-positive cancer as defined above. For example, the sample may be obtained from a tumorous tissue, (a) tumor(s) and, accordingly, is (a) tumor cell(s) or (a) tumor tissue(s) suspected of being HER2-positive tumour, like a breast tumor and the like. A person skilled in the art is in the position to identify such tumors and/or individuals/patients suffering from corresponding cancer using standard techniques known in the art and methods disclosed herein. Generally, said tumor cell or cancer cell may be obtained from any biological source/organism, particularly any biological source/organism, suffering from the above-mentioned cancer. In context of this invention particular useful cells are, preferably, human cells. These cells can be obtained from e.g. biopsies or from biological samples. The tumor/cancer/tumor cell/cancer cell is a solid tumor/cancer/tumor cell/cancer cell. In accordance with the above, the cancer/tumor cell may be a breast cancer/tumor cell or said sample comprises a cancer/tumor cell, such as a breast cancer/tumor cell. In line with the above, said tumor/cancer may be a breast tumor/cancer.

The modulator of the HER2/neu (ErbB2) signaling pathway may be an inhibitor of HER2, for example, a HER dimerization/signaling inhibitor. The HER dimerization inhibitor may be a HER2 dimerization inhibitor. The HER dimerization inhibitor may inhibit HER heterodimerization or HER homodimerization. The HER dimerization inhibitor may be an anti-HER antibody. The term “antibody” herein is used in the broadest sense and specifically covers intact monoclonal antibodies, polyclonal antibodies, multispecific antibodies (e.g., bispecific antibodies) formed from at least two intact antibodies, and antibody fragments, so long as they exhibit the desired biological activity. Also human and humanized as well as CDR-grafted antibodies are comprised within the term “antibody”.

The HER antibody may bind to a HER receptor selected from the group consisting of EGFR, HER2 and HER3. Preferably, the antibody binds to HER2. The anti HER2 antibody may bind to domain II of HER2 extracellular domain. The antibody may bind to a junction between domains I, II and III of HER2 extracellular domain. The anti HER2 antibody may be Pertuzumab.

For the purposes herein, “Pertuzumab” and “rhuMAb 2C4”, which are used interchangeably, refer to an antibody comprising the variable light and variable heavy domains (amino acid sequences thereof shown in SEQ ID Nos. 5 and 6, respectively, as depicted in FIG. 2). The variable light and variable heavy domains of variant 574/Pertuzumab are also shown in FIG. 2 (amino acid sequences thereof shown in SEQ ID Nos. 7 and 8, respectively, as depicted in FIG. 2). Where Pertuzumab is an intact antibody, it preferably comprises an IgG1 antibody; in one embodiment comprising the light chain amino acid sequence in it preferably comprises the light chain and heavy chain amino acid sequences, respectively, as shown in FIGS. 3A/3B and 5A/5B (FIG. 5A/5B show the light chain and heavy chain amino acid sequences of a variant Pertuzumab). The heavy chain amino acid sequences of Pertuzumab as shown in FIG. 3B may optionally comprise an additional amino acid “K” at position 449 at the C-terminus. The antibody is optionally produced by recombinant Chinese Hamster Ovary (CHO) cells. The terms “Pertuzumab” and “rhuMAb 2C4” herein cover biosimilar versions of the drug with the United States Adopted Name (USAN) or International Nonproprietary Name (INN): Pertuzumab. Again, corresponding sequences are shown in FIGS. 2 to 5.

The modulator of the HER2/neu (ErbB2) signaling pathway may be an inhibitor of HER shedding, for example a HER2 shedding inhibitor. The inhibitor of HER shedding may inhibit HER heterodimerization or HER homodimerization. Said inhibitor of HER shedding may be an anti-HER antibody.

The anti-HER antibody may bind to a HER receptor selected from the group consisting of EGFR, HER2 and HER3. Preferably, the antibody binds to HER2. The HER2 antibody may bind to sub-domain IV of the HER2 extracellular domain. Preferably, the HER2 antibody is Herceptin™/Trastuzumab.

For the purposes herein, “Herceptin™”/“Trastuzumab” and “rhuMAb4D5-8”, which are used interchangeably, refer to an antibody comprising the variable light domains and variable heavy domains (amino acid sequences thereof are shown in FIG. 4, respectively; the domain is indicated by arrows). Where Trastuzumab is an intact antibody, it preferably comprises an IgG1 antibody; in one embodiment comprising the light chain amino and the heavy chain amino acid sequence as shown in FIG. 4. The antibody is optionally produced by Chinese Hamster Ovary (CHO) cells. The terms “Trastuzumab” and “rhuMAb4D5-8” herein cover biosimilar versions of the drug with the United States Adopted Name (USAN) or International Nonproprietary Name (INN): Trastuzumab.

The inhibitor of programmed death ligand 1 (PD-L1) may be an antibody specifically binding to PD-L1 (anti-PD-L1 antibody).

Exemplary anti-PD-L1 antibodies are disclosed in WO 2010/077634 which is incorporated herein in its entirety. Corresponding exemplary anti-PD-L1 antibodies to be used in accordance with the present invention are described below.

The anti-PD-L1 antibody may comprise a heavy chain variable region polypeptide comprising an HVR-H1, HVR-H2 and HVR-H3 sequence, wherein:

(SEQ ID NO: 1)
(a) the HVR-H1 sequence is GFTFSX1SWIH;
(SEQ ID NO: 2)
(b) the HVR-H2 sequence is AWIX2PYGGSX3YYADSVKG;
(SEQ ID NO: 3)
(c) the HVR-H3 sequence is RHWPGGFDY;

further wherein: X1 is D or G; X2 is S or L; X3 is T or S. X1 may be D; X2 may be S and X3 may be T.

The polypeptide may further comprise variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4). The framework sequences may be derived from human consensus framework sequences. The framework sequences may be VH subgroup III consensus framework. One or more of the framework sequences may be the following:

(SEQ ID NO: 4)
HC-FR1 is EVQLVESGGGLVQPGGSLRLSCAAS
(SEQ ID NO: 5)
HC-FR2 is WVRQAPGKGLEWV
(SEQ ID NO: 6)
HC-FR3 is RFTISADTSKNTAYLQMNSLRAEDTAVYYCAR
(SEQ ID NO: 7)
HC-FR4 is WGQGTLVTVSA.

The heavy chain polypeptide may be in combination with a variable region light chain comprising an HVR-L1, HVR-L2 and HVR-L3, wherein:

(SEQ ID NOs: 8)
(a) the HVR-L1 sequence is RASQX4X5X6TX7X8A;
(SEQ ID NOs: 9)
(b) the HVR-L2 sequence is SASX9LX10S,
and;
(SEQ ID NOs: 10)
(c) the HVR-L3 sequence is QQX11X12X13X14PX15T;

further wherein: X4 is D or V; X5 is V or I; X6 is S or N; X7 is A or F; X8 is V or L; X9 is F or T; X10 is Y or A; X11 is Y, G, F, or S; X12 is L, Y, F or W; X13 is Y, N, A, T, G, F or I; X14 is H, V, P, T or I; X15 is A, W, R, P or T.

X4 may be D; X5 may be V; X6 may be S; X7 may be A; X8 may be V; X9 may be F; X10 may be Y; X11 may be Y; X12 may be L; X13 may be Y; X14 may be H; X15 may be A.

The polypeptide may further comprise variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4). The framework sequences may be derived from human consensus framework sequences. The framework sequences may be VL kappa I consensus framework. One or more of the framework sequences may be the following:

(SEQ ID NO: 11)
LC-FR1 is DIQMTQSPSSLSASVGDRVTITC;
(SEQ ID NO: 12)
LC-FR2 is WYQQKPGKAPKLLIY;
(SEQ ID NO: 13)
LC-FR3 is GVPSRFSGSGSGTDFTLTISSLQPEDFATYYC;
(SEQ ID NO: 14)
LC-FR4 is FGQGTKVEIKR.

The anti-PD-L1 antibody (or an antigen binding fragment thereof) may comprise a heavy chain and a light chain variable region sequence, wherein:
(a) the heavy chain comprises an HVR-H1, HVR-H2 and HVR-H3, wherein further:

(SEQ ID NO: 1)
(i) the HVR-H1 sequence is GFTFSX1SWIH;
(SEQ ID NO: 2)
(ii) the HVR-H2 sequence is AWIX2PYGGSX3YYADSVKG;
(SEQ ID NO: 3)
(iii) the HVR-H3 sequence is RHWPGGFDY,
and;

(b) the light chain comprises an HVR-L1, HVR-L2 and HVR-L3, wherein further:

(SEQ ID NOs: 8)
(iv) the HVR-L1 sequence is RASQX4X5X6TX7X8A;
(SEQ ID NOs: 9)
(v) the HVR-L2 sequence is SASX9LX10S;
(SEQ ID NOs: 10)
(vi) the HVR-L3 sequence is QQX11X12X13X14PX15T;

wherein: X1 is D or G; X2 is S or L; X3 is T or S; X4 may be D or V; X5 may be V or I; X6 may be S or N; X7 may be A or F; X8 may be V or L; X9 may be F or T; X10 may be Y or A; X11 may be Y, G, F, or S; X12 may be L, Y, F or W; X13 may be Y, N, A, T, G, F or I; X14 may be H, V, P, T or I; X15 may be A, W, R, P or T.

X1 may be D; X2 may be S and X3 may be T. Furthermore, the positions may be as follows: X4=D, X5=V, X6=S, X7=A and X8=V, X9=F, and X10=Y, X11=Y, X12=L, X13=Y, X14=H and/or X15=A. Furthermore, the positions may be as follows: X1=D, X2=S and X3=T, X4=D, X5=V, X6=S, X7=A and X8=V, X9=F, and X10=Y, X11=Y, X12=L, X13=Y, X14=H and X15=A.

The antibody (an antigen binding fragment thereof) may further comprise

(a) variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4), and

(b) variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4).

The framework sequences may be derived from human consensus framework sequences.

The variable region heavy chain framework sequences may be VH subgroup III consensus framework. One or more of the framework sequences may be the following:

(SEQ ID NO: 4)
HC-FR1 is EVQLVESGGGLVQPGGSLRLSCAAS;
(SEQ ID NO: 5)
HC-FR2 is WVRQAPGKGLEWV;
(SEQ ID NO: 6)
HC-FR3 is RFTISADTSKNTAYLQMNSLRAEDTAVYYCAR;
(SEQ ID NO: 7)
HC-FR4 is WGQGTLVTVSA.

The variable region light chain framework sequences may be VL kappa I consensus framework. One or more of the framework sequences may be the following:

(SEQ ID NO: 11)
LC-FR1 is DIQMTQSPSSLSASVGDRVTITC;
(SEQ ID NO: 12)
LC-FR2 is WYQQKPGKAPKLLIY;
(SEQ ID NO: 13)
LC-FR3 is GVPSRFSGSGSGTDFTLTISSLQPEDFATYYC,
and;
(SEQ ID NO: 14)
LC-FR4 is FGQGTKVEIKR.

The antibody (or antigen binding fragment thereof) may be or may comprise

(a) the variable heavy chain framework sequences are the following:

(SEQ ID NO: 4)
(i) HC-FR1 is EVQLVESGGGLVQPGGSLRLSCAAS;
(SEQ ID NO: 5)
(ii) HC-FR2 is WVRQAPGKGLEWV;
(SEQ ID NO: 6)
(iii) HC-FR3 is RFTISADTSKNTAYLQMNSLRAEDTAVYYCAR;
(SEQ ID NO: 7)
(iv) HC-FR4 is WGQGTLVTVSA;
and;

(b) the variable light chain framework sequences are the following:

(SEQ ID NO: 11)
(i) LC-FR1 is DIQMTQSPSSLSASVGDRVTITC;
(SEQ ID NO: 12)
(ii) LC-FR2 is WYQQKPGKAPKLLIY;
(SEQ ID NO: 13)
(iii) LC-FR3 is GVPSRFSGSGSGTDFTLTISSLQPEDFATYYC;
(SEQ ID NO: 14)
(iv) LC-FR4 is FGQGTKVEIKR.

The antibody (or fragment thereof) may further comprise a human constant region. The constant region may selected from the group consisting of IgG1, IgG2, IgG3 and IgG4. The constant region may be IgG1. The antibody (or fragment thereof) may further comprise murine constant region. The constant region may be selected from the group consisting of IgG1, IgG2A, IgG2B and IgG3. The constant region may be IgG2A.

The antibody (or fragment thereof) may have reduced or minimal effector function. The minimal effector function may result from an effector-less Fc mutation. The effector-less Fc mutation may be N297A. The effector-less Fc mutation may be D265A/N297A. The minimal effector function may result from aglycosylation.

The antibody (or fragment thereof) may comprise a heavy chain and a light chain variable region sequence, wherein:

(a) the heavy chain comprises an HVR-H1, HVR-H2 and an HVR-H3, having at least 85% overall sequence identity to GFTFSDSWIH (SEQ ID NO:15), AWISPYGGSTYYADSVKG (SEQ ID NO:16) and RHWPGGFDY (SEQ ID NO:3), respectively, and

(b) the light chain comprises an HVR-L1, HVR-L2 and an HVR-L3, having at least 85% overall sequence identity to RASQDVSTAVA (SEQ ID NO:17), SASFLYS (SEQ ID NO:18) and QQYLYHPAT (SEQ ID NO:19), respectively.

The sequence identity may be at least 90%.

The antibody (or fragment thereof) may further comprise:

(a) variable region heavy chain (VH) framework sequences juxtaposed between the HVRs according to the formula: (HC-FR1)-(HVR-H1)-(HC-FR2)-(HVR-H2)-(HC-FR3)-(HVR-H3)-(HC-FR4), and

(b) variable region light chain (VL) framework sequences juxtaposed between the HVRs according to the formula: (LC-FR1)-(HVR-L1)-(LC-FR2)-(HVR-L2)-(LC-FR3)-(HVR-L3)-(LC-FR4).

The antibody (or fragment thereof) may further comprise a VH and VL framework region derived from a human consensus sequence. The VH framework sequence may be derived from a Kabat subgroup I, II, or III sequence. The VH framework sequence may be a Kabat subgroup III consensus framework sequence. The VH framework sequences may be the following:

(SEQ ID NO: 4)
HC-FR1 is EVQLVESGGGLVQPGGSLRLSCAAS;
(SEQ ID NO: 5)
HC-FR2 is WVRQAPGKGLEWV;
(SEQ ID NO: 6)
HC-FR3 is RFTISADTSKNTAYLQMNSLRAEDTAVYYCAR;
(SEQ ID NO: 7)
HC-FR4 is WGQGTLVTVSA.

The VL framework sequence may be derived from a Kabat kappa I, II, III or IV subgroup sequence. The VL framework sequence may be a Kabat kappa I consensus framework sequence.

The VL framework sequences may be the following:

(SEQ ID NO: 11)
LC-FR1 is DIQMTQSPSSLSASVGDRVTITC;
(SEQ ID NO: 12)
LC-FR2 is WYQQKPGKAPKLLIY;
(SEQ ID NO: 13)
LC-FR3 is GVPSRFSGSGSGTDFTLTISSLQPEDFATYYC;
(SEQ ID NO: 14)
LC-FR4 is FGQGTKVEIKR.

The antibody (or fragment thereof) may comprise a heavy chain and a light chain variable region sequence, wherein:

(a) the heavy chain sequence has at least 85% sequence identity to the heavy chain sequence:

(SEQ ID NO: 20)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSA,

and

(b) the light chain sequence has at least 85% sequence identity to the light chain sequence:

(SEQ ID NO: 21)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

The sequence identity may be at least 90%.

The antibody (or fragment thereof) may comprise a heavy chain and light chain variable region sequence, wherein:

(a) the heavy chain comprises the sequence: EVQLVESGGGLVQPGGSLRLS CAASGFTFSDSWIHWVRQAPGKGLEWVAWISPYGGSTYYADSVKGRFTISADTSKN TAYLQMNSLRAEDTAVYYCARRHWPGGFDYWGQGTLVTVSA (SEQ ID NO:20), and

(b) the light chain comprises the sequence: DIQMTQSPSSLSASVGDRVTITC RASQDVSTAVAWYQQKPGKAPKLLIYSASFLYSGVPSRFSGSGSGTDFTLTISSLQPE DFATYYCQQYLYHPATFGQGTKVEIKR (SEQ ID NO:21).

Moreover, the anti-PD-L1 antibody may be encoded by a nucleic acid. Accordingly, herein described is an isolated nucleic acid encoding the above polypeptide/antibody (or fragment thereof).

Provided herein is an isolated nucleic acid encoding a light chain or a heavy chain variable sequence of an anti-PD-L1 antibody or antigen binding fragment, wherein:

(a) the heavy chain further comprises and HVR-H1, HVR-H2 and an HVR-H3 sequence having at least 85% sequence identity to GFTFSDSWIH (SEQ ID NO:15), AWISPYGGSTYYADSVKG (SEQ ID NO:16) and RHWPGGFDY (SEQ ID NO:3), respectively, or

(b) the light chain further comprises an HVR-L1, HVR-L2 and an HVR-L3 sequence having at least 85% sequence identity to RASQDVSTAVA (SEQ ID NO:17), SASFLYS (SEQ ID NO:18) and QQYLYHPAT (SEQ ID NO:19), respectively.

The sequence identity may be 90%. The anti-PD-L1 antibody may further comprise a VL and a VH framework region derived from a human consensus sequence. The VH sequence may be derived from a Kabat subgroup I, II, or III sequence. The VL sequence may be derived from a Kabat kappa I, II, III or IV subgroup sequence. The anti-PD-L1 antibody may comprise a constant region derived from a murine antibody. The anti-PD-L1 antibody may comprise a constant region derived from a human antibody. The constant region may be IgG1. The antibody encoded by the nucleic acid may have reduced or minimal effector function. The minimal effector function may result from an effector-less Fc mutation. The effector-less Fc mutation may be N297A.

Further provided herein is a vector comprising the nucleic acid, a host cell comprising the vector. The host cell may be eukaryotic. The host cell may be mammalian. The host cell may be a Chinese Hamster Ovary (CHO) cell. The host cell may be prokaryotic. The host cell may be E. coli. Also provided herein is a process for making an anti-PD-L1 antibody comprising culturing the above host cell under conditions suitable for the expression of the vector encoding the anti-PD-L1 antibody or antigen binding fragment, and recovering the antibody or fragment.

The following describes in more detail the herein provided means and methods for treating a cancer and/or a cancer patient.

Herein contemplated is, accordingly, a pharmaceutical composition comprising a modulator of the HER2/neu (ErbB2) signaling pathway (like Trastuzumab), and an inhibitor of programmed death ligand 1 (PD-L1) (like the anti-PD-L1 antibody described herein) for use in the treatment of cancer, whereby said cancer is determined to have a low or absent ER expression level and to have an increased expression level of programmed death ligand 1 (PD-L1) in comparison to a control. The cancer may be determined to have a decreased expression level of interferon-gamma (IFNγ) in comparison to the control. The pharmaceutical composition may further comprise a chemotherapeutic agent (like taxol or a taxol derivative, such as dodetaxel (Taxotere®)).

In accordance with the above, the present invention provides a method for treating cancer comprising administering an effective amount of a modulator of the HER2/neu (ErbB2) signaling pathway, a chemotherapeutic agent and an inhibitor of programmed death ligand 1 (PD-L1) to a subject in need thereof. The cancer may be determined to have a decreased expression level of interferon-gamma (IFNγ) in comparison to the control.

Herein provided is a modulator of the HER2/neu (ErbB2) signaling pathway, and an inhibitor of programmed death ligand 1 (PD-L1) for use in the treatment of cancer, whereby said cancer is determined to have a low or absent ER expression level and to have an increased expression level of programmed death ligand 1 (PD-L1) in comparison to a control. Moreover, herein provided is a modulator of the HER2/neu (ErbB2) signaling pathway, an inhibitor of programmed death ligand 1 (PD-L1) and a chemotherapeutic agent (like taxol or a taxol derivative, such as dodetaxel (Taxotere®)) for use in the treatment of cancer, whereby said cancer is determined to have a low or absent ER expression level and to have an increased expression level of programmed death ligand 1 (PD-L1) in comparison to a control. The cancer may be determined to have a decreased expression level of interferon-gamma (IFNγ) in comparison to the control.

As discussed above, the present invention provides a method of treating a cancer in a cancer patient for whom therapy comprising a modulator of the HER2/neu (ErbB2) signaling pathway and a chemotherapeutic agent is contemplated, the method comprising selecting a cancer patient whose cancer is determined to have a low or absent ER expression level and to have an increased expression level of programmed death ligand 1 (PD-L1) in comparison to a control, and administering to the patient an effective amount of a modulator of the HER2/neu (ErbB2) signaling pathway, of a chemotherapeutic agent and of a programmed death ligand 1 (PD-L1) inhibitor. Likewise, the present invention provides a method of treating a cancer in a cancer patient who is undergoing therapy comprising a modulator of the HER2/neu (ErbB2) signaling pathway and a chemotherapeutic agent, the method comprising selecting a cancer patient whose cancer is determined to have a low or absent ER expression level and to have an increased expression level of programmed death ligand 1 (PD-L1) in comparison to a control, and administering to the patient an effective amount of a programmed death ligand 1 (PD-L1) inhibitor.

The explanations and definitions given herein above in relation to “cancer”, “cancer patient”, “PD-L1 inhibitor”, “PD-L1 inhibitor therapy”, “modulator of the HER2/neu (ErbB2) signaling pathway”, “chemotherapeutic agent”, “low or absent ER expression level” “increased expression level of programmed death ligand 1 (PD-L1)”, “decreased expression level of interferon-gamma (IFN-γ) and the like apply, mutatis mutandis, in the context of the herein.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a patient and includes: (a) preventing a disease related in a patient which may be predisposed to the disease; (b) inhibiting the disease, i.e. arresting its development; or (c) relieving the disease, i.e. causing regression of the disease.

A “patient” for the purposes of the present invention includes both humans and other animals, particularly mammals, and other organisms. Thus, the methods are applicable to both human therapy and veterinary applications. Preferably, the patient is human.

The below explanations relate in more detail to the treatment/therapy of these patients/this patient group in accordance with the present invention.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

The skilled person knows that the effective amount of one of the herein described PD-L1 inhibitor(s), modulator(s) of the HER2/neu (ErbB2) signaling pathway and chemotherapeutic agent(s) in a pharmaceutical composition administered to an individual will, inter alia, depend on the nature of the compound. For example, if said compound is a (poly)peptide or protein the total pharmaceutically effective amount of pharmaceutical composition administered parenterally per dose will be in the range of about 1 μg protein/kg/day to 10 mg protein/kg/day of patient body weight, although, as noted above, this will be subject to therapeutic discretion. More preferably, this dose is at least 0.01 mg protein/kg/day, and most preferably for humans between about 0.01 and 1 mg protein/kg/day.

The following administration may be employed in respect of Trastuzumab:

Posology and Method of Administration

HER2 testing is mandatory prior to initiation of therapy. Herceptin treatment should only be initiated by a physician experienced in the administration of cytotoxic chemotherapy.

MBC

Three-Weekly Schedule

The recommended initial loading dose is 8 mg/kg body weight. The recommended maintenance dose at three-weekly intervals is 6 mg/kg body weight, beginning three weeks after the loading dose.

Weekly Schedule

The recommended initial loading dose of Herceptin is 4 mg/kg body weight. The recommended weekly maintenance dose of Herceptin is 2 mg/kg body weight, beginning one week after the loading dose.

Administration in Combination with Paclitaxel or Docetaxel

In the pivotal trials (H0648g, M77001), paclitaxel or docetaxel was administered the day following the first dose of Herceptin (for dose, see the Summary of Product Characteristics for paclitaxel or docetaxel) and immediately after the subsequent doses of Herceptin if the preceding dose of Herceptin was well tolerated.

Administration in Combination with an Aromatase Inhibitor

In the pivotal trial (BO16216) Herceptin and anastrozole were administered from day 1. There were no restrictions on the relative timing of Herceptin and anastrozole at administration (for dose, see the Summary of Product Characteristics for anastrozole or other aromatase inhibitors).

EBC

Three-Weekly and Weekly Schedule

As a three-weekly regimen the recommended initial loading dose of Herceptin is 8 mg/kg body weight. The recommended maintenance dose of Herceptin at three-weekly intervals is 6 mg/kg body weight, beginning three weeks after the loading dose.

As a weekly regimen (initial loading dose of 4 mg/kg followed by 2 mg/kg every week) concomitantly with paclitaxel following chemotherapy with doxorubicin and cyclophosphamide.

(See section 5.1 for chemotherapy combination dosing).

MGC

Three-Weekly Schedule

The recommended initial loading dose is 8 mg/kg body weight. The recommended maintenance dose at three-weekly intervals is 6 mg/kg body weight, beginning three weeks after the loading dose.

Breast Cancer (MBC and EBC) and Gastric Cancer (MGC)

Duration of Treatment

Patients with MBC or MGC should be treated with Herceptin until progression of disease. Patients with EBC should be treated with Herceptin for 1 year or until disease recurrence, whatever occurs first.

Dose Reduction

No reductions in the dose of Herceptin were made during clinical trials. Patients may continue therapy during periods of reversible, chemotherapy-induced myelosuppression but they should be monitored carefully for complications of neutropenia during this time. Refer to the Summary of Product Characteristics for paclitaxel, docetaxel or aromatase inhibitor for information on dose reduction or delays.

Missed Doses

If the patient misses a dose of Herceptin by one week or less, then the usual maintenance dose (weekly regimen: 2 mg/kg; three-weekly regimen: 6 mg/kg) should be given as soon as possible. Do not wait until the next planned cycle. Subsequent maintenance doses (weekly regimen: 2 mg/kg; three-weekly regimen: 6 mg/kg respectively) should then be given according to the previous schedule.

If the patient misses a dose of Herceptin by more than one week, a re-loading dose of Herceptin should be given over approximately 90 minutes (weekly regimen: 4 mg/kg; three-weekly regimen: 8 mg/kg). Subsequent Herceptin maintenance doses (weekly regimen: 2 mg/kg; three-weekly regimen 6 mg/kg respectively) should then be given (weekly regimen: every week; three-weekly regimen every 3 weeks) from that point.

Special Patient Populations

Clinical data show that the disposition of Herceptin is not altered based on age or serum creatinine In clinical trials, elderly patients did not receive reduced doses of Herceptin. Dedicated pharmacokinetic studies in the elderly and those with renal or hepatic impairment have not been carried out. However, in a population pharmacokinetic analysis, age and renal impairment were not shown to affect trastuzumab disposition.

Method of Administration

Herceptin loading dose should be administered as a 90-minute intravenous infusion. Do not administer as an intravenous push or bolus. Herceptin intravenous infusion should be administered by a health-care provider prepared to manage anaphylaxis and an emergency kit should be available. Patients should be observed for at least six hours after the start of the first infusion and for two hours after the start of the subsequent infusions for symptoms like fever and chills or other infusion-related symptoms (see sections 4.4 and 4.8). Interruption or slowing the rate of the infusion may help control such symptoms. The infusion may be resumed when symptoms abate.

If the initial loading dose was well tolerated, the subsequent doses can be administered as a 30-minute infusion. Pharmaceutical compositions of the invention may be administered parenterally.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion. The administration of the herein provided compositions may, inter alia, comprise an administration twice daily, every day, every other day, every third day, every fourth day, every fifth day, once a week, once every second week, once every third week, once every month, etc.

The pharmaceutical composition is also suitably administered by sustained release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained release pharmaceutical composition also include liposomally entrapped compound. Liposomes containing the pharmaceutical composition are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The components of the pharmaceutical composition to be used for therapeutic administration must be sterile. Sterility is readily accomplished by filtration through sterile filtration membranes (e.g., 0.2 micron membranes). Therapeutic components of the pharmaceutical composition generally are placed into a container having a sterile access port, for example, an intravenous solution bag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

The herein provided treatment of cancer comprising a modulator of the HER2/neu (ErbB2) signaling pathway, an inhibitor of programmed death ligand 1 (PD-L1) and a chemotherapeutic agent (like taxol or a taxol derivative, such as dodetaxel (Taxotere®)) may be performed by way of the simultaneous, sequential or separate administration of the individual components of said treatment. For example, one or more of the modulator(s) of the HER2/neu (ErbB2) signaling pathway as defined herein (like Trastuzumab) may be administered simultaneously with one or more of the herein defined inhibitor(s) of programmed death ligand 1 (PD-L1) (like the herein provided and described anti-PD-L1 antibodies). Also, sequential administration of the modulator(s) of the HER2/neu (ErbB2) signaling pathway as defined herein (like Trastuzumab) may be administered simultaneously with one or more of the herein defined inhibitor(s) of programmed death ligand 1 (PD-L1) (like the herein provided and described anti-PD-L1 antibodies) to be used in accordance with the present invention is envisaged herein. The herein defined modulators of the HER2/neu (ErbB2) signaling pathway as defined herein (like Trastuzumab) and the one or more of the herein defined inhibitor of programmed death ligand 1 (PD-L1) (like the herein provided and described anti-PD-L1 antibodies) may also be administered separately. For example, one or more of the modulator(s) of the HER2/neu (ErbB2) signaling pathway as defined herein (like Trastuzumab) may be administered in a first step followed by administration in a second step with one or more of the inhibitor(s) of programmed death ligand 1 (PD-L1) (like the herein provided and described anti-PD-L1 antibodies) and vice versa. Likewise, the chemotherapeutic agent may be administered simultaneously, sequentially or separately. Any combination of simultaneous, sequential or separate administration of the modulator(s) of the HER2/neu (ErbB2) signaling pathway, inhibitor(s) of programmed death ligand 1 (PD-L1) and chemotherapeutic agent(s) (like taxol or a taxol derivative, such as dodetaxel (Taxotere®)) is envisaged herein.

The herein provided treatment of cancer comprising a modulator of the HER2/neu (ErbB2) signaling pathway, an inhibitor of programmed death ligand 1 (PD-L1) and a chemotherapeutic agent (like taxol or a taxol derivative, such as dodetaxel (Taxotere®)) can be applied as a sole therapy. It may, however, also be applied with one or more additional therapies (i.e. in a further cotherapy with), for example, conventional therapies like surgery, radiotherapy and/or one or more additional chemotherapeutic agents.

Surgery may comprise the step of partial or complete tumour resection, prior to, during or after the administration of the herein provided cancer treatment comprising a modulator of the HER2/neu (ErbB2) signaling pathway, an inhibitor of programmed death ligand 1 (PD-L1) and a chemotherapeutic agent (like taxol or a taxol derivative, such as dodetaxel (Taxotere®)). The herein provided modulator of the HER2/neu (ErbB2) signaling pathway, inhibitor of programmed death ligand 1 (PD-L1) and chemotherapeutic agent (like taxol or a taxol derivative, such as dodetaxel (Taxotere®)) may be administered in a neoadjuvant or adjuvant setting (in particular neoadjuvant or adjuvant treatment of cancer).

The modulator of the HER2/neu (ErbB2) signaling pathway, the chemotherapeutic agent and the inhibitor of programmed death ligand 1 (PD-L1) can be administered in a neoadjuvant setting. The modulator of the HER2/neu (ErbB2) signaling pathway, the chemotherapeutic agent and the inhibitor of programmed death ligand 1 (PD-L1) can be administered in an adjuvant setting or in a metastatic setting.

Accordingly, the herein provided modulator of the HER2/neu (ErbB2) signaling pathway, an inhibitor of programmed death ligand 1 (PD-L1) and a chemotherapeutic agent (like taxol or a taxol derivative, such as dodetaxel (Taxotere®)) may be administered to a patient in need of such a treatment during or after a surgical intervention/resection of the cancerous tissue. Therefore, the present invention is useful in neoadjuvant therapy, i.e. the treatment with the herein provided therapy given to a patient/patient group in need thereof prior to surgery. It is also useful in adjuvant therapy (i.e. after surgery).

The chemotherapeutic agent to be used herein is preferably a taxane (the term “taxol” is used interchangeably herein with “taxane”) or a taxane derivate (taxol derivative), like dodetaxel (Taxotere®) or paclitaxel. The use of dodetaxel/(Taxotere®) is particularly preferred herein.

The (additional) chemotherapeutic agent(s) may be one or more of the following exemplary, non-limiting, drugs or agents:

Cisplatin, Vinorelbin, Carboplatin, Paclitaxel, Gemcitabin, Docetaxel, Bevacizumab, Pemetrexed, Etoposid, Irinotecan, Ifosfamid, Topotecan,

(an) anti-angiogenic agent(s) like a VEGF blocker (such as bevacizumab/Avastin or sutent (sunitinib malate-SU-11248)), linomide, inhibitors of integrin αvβ3 function, angiostatin, razoxin, thalidomide, and including vascular targeting agents (for example combretastatin phosphate or N-acetylcolchinol-O-phosphate));

(an) cytostatic agent(s) such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example platelet derived growth factor and hepatocyte growth factor such inhibitors include growth factor antibodies, growth factor receptor antibodies, tyrosine kinase inhibitors and serine/threonine kinase inhibitors);

biological response modifiers (for example interferon); (an) anti-metabolite agent(s) (for example gemcitabine); (an) anti-hormonal compound(s) such as (an) anti-estrogen(s); antibodies (for example edrecolomab); adjuvant (anti-) hormonal therapy/therapies (i.e. therapy with (an) adjuvant (anti-) hormone drug(s), such as tamoxifen; gene therapy approaches (like antisense therapies); and/or immunotherapy approaches.

The chemotherapy may also (additionally) include the use of one or more of antiproliferative/antineoplastic drugs and combinations thereof, as used in medical oncology, such as (an) tyrosine kinase inhibitor(s), (a) raf inhibitor(s), (a) ras inhibitor(s), (a) dual tyrosine kinase inhibitor(s), taxol, (an) taxane(s) (like paclitaxel or docetaxel), (an) anthracycline(s), like doxorubicin or epirubicin, aromatase inhibitors (such as anastrozole or letrozole) and/or vinorelbine; cyclophosphamide, methotrexate or fluorouracil (which is also known as 5-FU) can be used in such cotherapy individually or in form of a cotherapy comprising these three drugs (“CMF therapy”), optionally in combination with any of the other herein provided additional therapies. Particular examples of chemotherapeutic agents for use with a combination treatment of the present invention are pemetrexed, raltitrexed, etoposide, vinorelbine, paclitaxel, docetaxel, cisplatin, oxaliplatin, carboplatin, gemcitabine, irinotecan (CPT-1 1), 5-fluorouracil (5-FU, (including capecitabine)), doxorubicin, cyclophosphamide, temozolomide, hydroxyurea, (iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine and taxoids like taxol, taxotere); topoisomerase inhibitors (for example epipodophyllotoxins like etoposide and teniposide, amsacrine, topotecan, and also irinotecan); also enzymes (for example asparaginase); and thymidylate synthase inhibitors (for example raltitrexed); and additional types of chemotherapeutic agents.

Inhibitors/Modulators/chemotherapeutic agents for use in accordance with the present invention are described herein and refer generally to known and/or commercially available Inhibitors/Modulators/chemotherapeutic. However, the use of inhibitors yet to be generated or known compounds to be tested for their inhibiting activity is envisaged in context of the present invention.

In a further aspect, the present invention relates to the use of (a) nucleic acid(s) or antibody (antibodies) capable of detecting the expression level of ER, PD-L1 and, optionally, IFNγ for determining a patient's need for PD-L1 inhibitor cotherapy in combination with a modulator of the HER2/neu (ErbB2) signaling pathway and a chemotherapeutic agent. The respective explanations of said terms have been given above and apply here mutatis mutandis.

Preferably, the nucleic acid (e.g. oligonucleotide(s)) is (are) about 15 to 100 nucleotides in length. A person skilled in the art is, based on his general knowledge and the teaching provided herein, easily in the position to identify and/or prepare (a) an oligo- or polynucleotide capable of detecting the expression level of ER, PD-L1 and, optionally, IFNγ. In particular these nucleic acid(s) (e.g. oligo- or polynucleotides) may be used as probe(s) in the methods described herein, for example in the measurement of the expression level. A skilled person will know, for example, computer programs which may be useful for the identification of corresponding probes to be used herein. For example, a nucleic acid encoding estrogen receptor (or a part of the nucleic acid) (e.g. SEQ ID NO: 38), a nucleic acid encoding PD-L1 (or a part of the nucleic acid) (e.g. SEQ ID NO: 42) and, optionally, a nucleic acid encoding IFNγ (or a part of the nucleic acid) (e.g. SEQ ID NO: 44 may be used in this context for identifying specific probes for detecting the expression level of ER, PD-L1 and IFNγ, respectively. Exemplary nucleic acid sequences encoding ER, PD-L1 and IFNγ are available on corresponding databases, such as the NCBI database (world wide web at ncbi.nlm.nih.gov/sites/entrez).

Furthermore, a composition is provided herein which is a diagnostic composition further comprising, optionally, means for detection/determining/evaluating the expression level of ER, PD-L1 and IFNγ. Such means for detection, are, for example, the above-described nucleotides and/or antibodies. Accordingly, the present invention relates to such means (e.g. such nucleotides and/or antibodies) for the preparation of a diagnostic composition for determining a patient in need of a PD-L1 inhibitor cotherapy.

In an alternative aspect, the present invention relates to such means for detection (e.g the above-described nucleic acids and/or antibodies and/or the “binding molecules” described below in context of the kit to be used in accordance with the present invention) for use in determining a patient in need of a PD-L1 inhibitor cotherapy. Preferably, the present invention relates to (an) antibody/antibodies for use in determining a patient in need of a PD-L1 inhibitor cotherapy.

Furthermore, the present invention also relates to a kit useful for carrying out the herein provided methods, the kit comprising (a) nucleic acid or (an) antibody capable of detecting the expression level of ER, PD-L1 and, optionally, IFNγ. Also envisaged herein is the use of the herein described kit for carrying out the herein provided methods. Said kit useful for carrying out the methods and uses described herein may comprise oligonucleotides or polynucleotides capable of determining the expression level of ER, PD-L1 and, optionally, IFNγ. For example, said kit may comprise (a) compound(s) required for specifically measuring the expression level of ER, PD-L1 and, optionally, IFNγ. Moreover, the present invention also relates to the use of (a) compound(s) required for specifically measuring the expression level of ER, PD-L1 and, optionally, IFNγ, for the preparation of a kit for carrying out the methods or uses of this invention. On the basis of the teaching of this invention, the skilled person knows which compound(s) is (are) required for specifically measuring the expression level of ER, PD-L1 and, optionally, IFNγ. For example, such compound(s) may be (a) “binding molecule(s)”. Particularly, such compound(s) may be (a) (nucleotide) probe(s), (a) primer(s) (pair(s)), (an) antibody(ies) and/or (an) aptamer(s) specific for a (gene) product of the ER gene/coding sequence, PD-L1 gene/coding sequence and, optionally, IFNγ/coding sequence. The kit (to be prepared in context) of this invention may be a diagnostic kit.

The kit (to be prepared in context) of this invention or the methods and uses of the invention may further comprise or be provided with (an) instruction manual(s). For example, said instruction manual(s) may guide the skilled person (how) to determine the (reference/control) expression level of ER, PD-L1 and, optionally, IFNγ. or (how) to determine a patient's need of PD-L1 inhibitor therapy. Particularly, said instruction manual(s) may comprise guidance to use or apply the herein provided methods or uses. The kit (to be prepared in context) of this invention may further comprise substances/chemicals and/or equipment suitable/required for carrying out the methods and uses of this invention. For example, such substances/chemicals and/or equipment are solvents, diluents and/or buffers for stabilizing and/or storing (a) compound(s) required for specifically measuring the expression level of ER, PD-L1 and, optionally, IFNγ.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of.” Thus, the terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method. Thus, the term “consisting essentially of” means that specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

As used herein, the term “isolated” refers to a composition that has been removed from its in-vivo location (e.g. aquatic organism or moss). Preferably the isolated compositions of the present invention are substantially free from other substances (e.g., other proteins that do not comprise anti-adhesive effects) that are present in their in-vivo location (i.e. purified or semi-purified).

As used herein the term “about” refers to ±10%.

The present invention also relates to the following items:
1. A method of determining the need of a cancer patient for a PD-L1 inhibitor cotherapy,
   (i) wherein therapy comprising a modulator of the HER2/neu (ErbB2) signaling pathway and a chemotherapeutic agent is contemplated for the patient or (ii) wherein the patient is undergoing therapy comprising a modulator of the HER2/neu (ErbB2) signaling pathway and a chemotherapeutic agent, said method comprising the steps of
   a) measuring in vitro in a sample from said patient the expression level of Estrogen receptor (ER) and of programmed death ligand 1 (PD-L1),
   b) determining a patient as being in need of a PD-L1 inhibitor cotherapy if a low or absent ER expression level and an expression level of programmed death ligand 1 (PD-L1) that is increased in comparison to a control is measured in step (a).
2. A method of treating a cancer in a cancer patient for whom therapy comprising a modulator of the HER2/neu (ErbB2) signaling pathway and a chemotherapeutic agent is contemplated, the method comprising selecting a cancer patient whose cancer is determined to have a low or absent ER expression level and to have an increased expression level of programmed death ligand 1 (PD-L1) in comparison to a control, and administering to the patient an effective amount of a modulator of the HER2/neu (ErbB2) signaling pathway, of a chemotherapeutic agent and of a programmed death ligand 1 (PD-L1) inhibitor.
3. A method of treating a cancer in a cancer patient who is undergoing therapy comprising a modulator of the HER2/neu (ErbB2) signaling pathway and a chemotherapeutic agent, the method comprising selecting a cancer patient whose cancer is determined to have a low or absent ER expression level and to have an increased expression level of programmed death ligand 1 (PD-L1) in comparison to a control, and administering to the patient an effective amount of a programmed death ligand 1 (PD-L1) inhibitor.
4. A pharmaceutical composition comprising a modulator of the HER2/neu (ErbB2) signaling pathway, and an inhibitor of programmed death ligand 1 (PD-L1) for use in the treatment of cancer, whereby said cancer is determined to have a low or absent ER expression level and to have an increased expression level of programmed death ligand 1 (PD-L1) in comparison to a control.
5. The pharmaceutical composition for use in the treatment of cancer of item 4, further comprising a chemotherapeutic agent.
6. The method of any one of items 1 to 3, further comprising measuring in vitro in a sample from said patient the expression level of interferon-gamma (IFNγ) and determining a patient as being in need of a PD-L1 inhibitor cotherapy if an expression level of interferon-gamma (IFNγ) that is decreased in comparison to a control is measured.
7. The method of any one of items 1, 2, 3 and 6; or the pharmaceutical composition of item 4 and 5, wherein the ER expression level is ER(−).
8. The method of any one of items 1, 2, 3, 6 and 7; or the pharmaceutical composition of any one of item 4, 5 and 7, wherein said modulator of the HER2/neu (ErbB2) signaling pathway is the HER2 antibody Herceptin/Trastuzumab.
9. The method of any one of items 1, 2, 3, 6, 7 and 8; or the pharmaceutical composition of any one of items 5, 7 and 8, wherein said chemotherapeutic agent is taxol or a taxol derivative.
10. The method of any one of items 1, 2, 3, 6, 7 and 8 to 9; or the pharmaceutical composition of any one of items 4, 5 and 7 to 9, wherein said inhibitor of programmed death ligand 1 (PD-L1) is an antibody specifically binding to PD-L1 (anti-PD-L1 antibody).
11. The method of any one of items 1, 2, 3, and 6 to 10; or the pharmaceutical composition of any one of items 4, 5 and 7 to 10, wherein said cancer is a solid cancer.
12. The method of item 11; or the pharmaceutical composition of item 11, wherein said solid cancer is breast cancer orgastric cancer
13. The method of any one of items 1, 2, 3, and 6 to 12; or the pharmaceutical composition of any one of items 4, 5 and 7 to 12, wherein the expression level of PD-L1 is the mRNA expression level.
14. Use of a nucleic acid or antibody capable of detecting the expression level of ER, PD-L1 and, optionally, IFNγ for determining a patient's need for PD-L1 inhibitor cotherapy in combination with a modulator of the HER2/neu (ErbB2) signaling pathway and a chemotherapeutic agent.
15. The method of any one of items 1, 2, 3 and 6 to 14; or the pharmaceutical composition of any one of items 4, 5 and 7 to 14, wherein said modulator of the HER2/neu (ErbB2) signaling pathway, said chemotherapeutic agent and said inhibitor of programmed death ligand 1 (PD-L1) are to be administered in a neoadjuvant setting.

The present invention is further described by reference to the following non-limiting figures and examples. Unless otherwise indicated, established methods of recombinant gene technology were used as described, for example, in Sambrook, Russell “Molecular Cloning, A Laboratory Manual”, Cold Spring Harbor Laboratory, N.Y. (2001) which is incorporated herein by reference in its entirety.

### BRIEF DESCRIPTION OF THE DRAWINGS

FIG. 1 provides a schematic of the HER2 protein structure, and amino acid sequences for Domains I-IV, respectively) of the extracellular domain thereof (SEQ ID NOS. 22-25, respectively, in order of appearance).

FIGS. 2A and 2B depict alignments of the amino acid sequences of the variable light (V_{L}) (FIG. 2A) and variable heavy (V_{H}) (FIG. 2B) domains of murine monoclonal antibody 2C4 (SEQ ID Nos. 26 and 27, respectively); V_{L} and V_{H} domains of variant 574/Pertuzumab (SEQ ID Nos. 28 and 29, respectively), and human V_{L} and V_{H} consensus frameworks (hum κ1, light kappa subgroup I; humIII, heavy subgroup III) (SEQ ID Nos. 30 and 31, respectively). Asterisks identify differences between variable domains of Pertuzumab and murine monoclonal antibody 2C4 or between variable domains of Pertuzumab and the human framework. Complementarity Determining Regions (CDRs) are in brackets.

FIGS. 3A and 3B show the amino acid sequences of Pertuzumab light chain (FIG. 3A; SEQ ID NO: 32) and heavy chain (FIG. 3B; SEQ ID NO: 33). CDRs are shown in bold. Calculated molecular mass of the light chain and heavy chain are 23,526.22 Da and 49,216.56 Da (cysteines in reduced form). The carbohydrate moiety is attached to Asn 299 of the heavy chain.

FIGS. 4A and 4B show the amino acid sequences of Trastuzumab light chain (FIG. 4A; SEQ ID NO: 34) and heavy chain (FIG. 4B; SEQ ID NO: 35), respectively. Boundaries of the variable light and variable heavy domains are indicated by arrows.

FIGS. 5A and 5B depict a variant Pertuzumab light chain sequence (FIG. 5A; SEQ ID NO: 36) and a variant Pertuzumab heavy chain sequence (FIG. 5B; SEQ ID NO: 37), respectively.

FIGS. 6A and 6B show known mRNA transcripts and position of the relevant AFFYMETRIX probe set target regions for gene CD274. Exons are shown as grey bold rectangles, junction regions are indicated by thin horizontal lines. Probe sets with their sequence mapped against mRNA sequences are shown as black bold rectangles. Provided coordinates are genomic coordinates on chromosome 9.

FIGS. 7A and 7B show known mRNA transcripts and position of the relevant AFFYMETRIX probe set target regions for gene IFNG. Exons are shown as grey bold rectangles, junction regions are indicated by thin horizontal lines. Probe sets with their sequence mapped against mRNA sequences are shown as black bold rectangles. Provided coordinates are genomic coordinates on chromosome 12.

FIG. 8 shows the distribution of the expression of genes IFNG and CD274 in the samples of ER- and ER-30 populations. Symbol types correspond to the final pCR status (solid: pCR achieved, open—pCR not achieved).

FIG. 9A is a box plot of expression of gene CD274 for ER− responders (pCR=YES) and nonresponders (pCR=NO). On the right the histograms of expression for both categories are provided. FIG. 9B shows a distribution of t-test statistics (HO hypothesis of no difference). The vertical mark indicates the actual value found in the involved sample. The area of the shaded regions corresponds to the alpha level.

FIG. 10A is a box plot of expression of gene IFNG for ER− responders (pCR=YES) and nonresponders (pCR=NO). On the right the histograms of expression for both categories are provided. FIG. 10B shows the distribution of t-test statistics (HO hypothesis of no difference). The vertical mark indicates the actual value found in the involved sample. The area of the shaded regions corresponds to the alpha level.

FIG. 11A is a box plot of expression of gene CD274 for ER+ responders (pCR=YES) and nonresponders (pCR=NO). On the right the histograms of expression for both categories are provided. FIG. 11B shows a distribution of t-test statistics (HO hypothesis of no difference). The vertical mark indicates the actual value found in the involved sample. The area of the shaded regions corresponds to the alpha level.

FIG. 12A is a box plot of expression of gene IFNG for ER+ responders (pCR=YES) and nonresponders (pCR=NO). On the right the histograms of expression for both categories are provided. FIG. 12B shows a distribution of t-test statistics (HO hypothesis of no difference). The vertical mark indicates the actual value found in the involved sample. The area of the shaded regions corresponds to the alpha level.

FIG. 13 is the receiver operating characteristic of the final logistic regression model for ER-population. Positive level is taken to be the positive response status (pCR=YES).

FIG. 14 is a LIFT curve of the final logistic regression model for ER− population. Positive level is taken to be the positive response status (pCR=YES). The Y-axis displays the ratio of how rich the portion of the population is in the chosen response level (upper curve corresponds to pCR=YES) compared to the rate of that response level as a whole.

FIG. 15 is an example of predicted clinical response status for ER− population. Shown is predicted profile of response as controlled by patient age, Cancer type, pN status, and expression of both genes involved. The actual predicted pCR probability (which is equal to 0.443) is given for NO LABC patient around 60 y. old and with expression in both genes around median values.

FIG. 16 is the receiver operating characteristic of the final logistic regression model for ER+ population. Positive level is taken to be the positive response status (pCR=YES).

FIG. 17 shows the distribution of age of ER− patients.

FIG. 18 shows the distribution of age of ER+ patients.

FIG. 19 shows a decision tree view on expression of IFNG and CD274 genes predicting clinical response in ER patients. The first two splits required to explain pCR are the ones wrt to IFNG and CD274.

The Example illustrates the invention.

### EXAMPLE 1: CANCER PATIENTS UNDERGOING HER2 TARGETED THERAPY AND CHEMOTHERAPY BENEFIT FROM PD-L1 INHIBITOR COTHERAPY, IF THE EXPRESSION LEVEL OF ER IS LOW OR ABSENT (ER NEGATIVE) AND IF PD-L1 EXPRESSION LEVEL IS INCREASED

Estimation of gene expression was performed with the help of R Bioconductor package ‘affy’, R version 2.15.0. All exploratory analyses and predictive models were made using SAS JMP ver. 10.0

48 HER2+, ER+ and 39 HER2+, ER− breast cancer biopsies were obtained from NeoSphere clinical trial. The samples had been taken at diagnosis from patients afterwards treated with Docetaxel and Trastuzumab in a neo-adjuvant setting. The distribution of main clinical covariates at base line, as well as of clinical response (as assessed at the surgery) in the involved population is as follows:

ER Negative Samples:

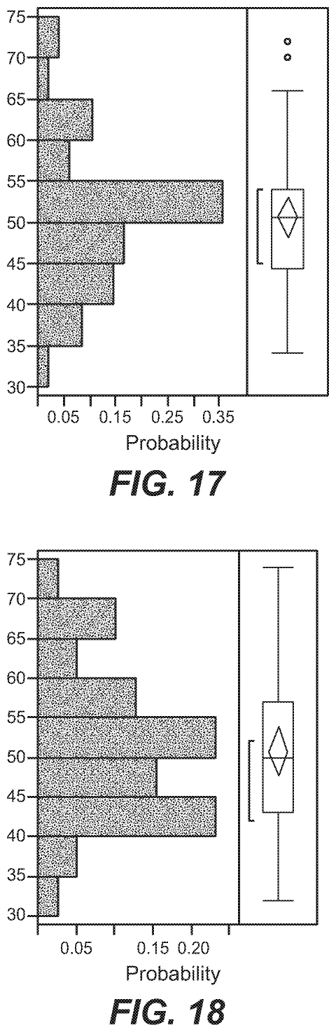

Patient Age (see FIG. 17)

Quantiles

| | | |
|---|---|---|
| 100.0% | maximum | 72 |
| 99.5% | | 72 |
| 97.5% | | 71.55 |
| 90.0% | | 64 |
| 75.0% | quartile | 54 |
| 50.0% | median | 50.5 |
| 25.0% | quartile | 44.25 |
| 10.0% | | 39 |
| 2.5% | | 34.675 |
| 0.5% | | 34 |
| 0.0% | minimum | 34 |

Cancer Type

| Level | Count | Prob |
|---|---|---|
| IBC | 2 | 0.04167 |
| LABC | 22 | 0.45833 |
| OPERABLE | 24 | 0.50000 |
| Total | 48 | 1.00000 |

pT (pathologic staging of Tumor)

| Level | Count | Prob |
|---|---|---|
| T2 | 18 | 0.37500 |
| T3 | 15 | 0.31250 |
| T4 | 15 | 0.31250 |
| Total | 48 | 1.00000 |

pN (pathologic staging of nodes)

| Level | Count | Prob |
|---|---|---|
| N0 | 12 | 0.25000 |
| N1 | 36 | 0.75000 |
| Total | 48 | 1.00000 |

G (Grade)

| Level | Count | Prob |
|---|---|---|
| G1 | 1 | 0.02083 |
| G2 | 15 | 0.31250 |
| G3 | 16 | 0.33333 |
| NA | 16 | 0.33333 |
| Total | 48 | 1.00000 |

ER Positive Samples:

Patient Age (see FIG. 18)

Quantiles

| | | |
|---|---|---|
| 100.0% | maximum | 74 |
| 99.5% | | 74 |
| 97.5% | | 74 |
| 90.0% | | 65 |
| 75.0% | quartile | 57 |
| 50.0% | median | 50 |
| 25.0% | quartile | 43 |
| 10.0% | | 40 |
| 2.5% | | 32 |
| 0.5% | | 32 |
| 0.0% | minimum | 32 |

Cancer Type

| Level | Count | Prob |
|---|---|---|
| IBC | 5 | 0.12821 |
| LABC | 8 | 0.20513 |
| OPERABLE | 26 | 0.66667 |
| Total | 39 | 1.00000 |

pT

| Level | Count | Prob |
|---|---|---|
| T2 | 15 | 0.38462 |
| T3 | 16 | 0.41026 |
| T4 | 8 | 0.20513 |
| Total | 39 | 1.00000 |

pN

| Level | Count | Prob |
|---|---|---|
| N0 | 11 | 0.28205 |
| N1 | 28 | 0.71795 |
| Total | 39 | 1.00000 |

G

| Level | Count | Prob |
|---|---|---|
| G2 | 13 | 0.33333 |
| G3 | 10 | 0.25641 |
| NA | 16 | 0.41026 |
| Total | 39 | 1.00000 |

Contingency Analysis of pathological complete response (pCR) By estrogen receptor status (ER)

| Count | | | |
|---|---|---|---|
| Row % | pCR = NO | pCR = YES | |
| ER = ER− | 27 | 21 | 48 |
| | 56.25 | 43.75 | |
| ER = ER+ | 33 | 6 | 39 |
| | 84.62 | 15.38 | |
| | 60 | 27 | 87 |

Gene Expression Profiling

The tumor biopsy samples were profiled for gene expression on AFFYMETRIX HG-U133Plus 2 whole Human Genome microarray platform. Roche HighPure RNA extraction, NuGen amplification and standard AFFYMETRIX hybridization and scanning protocols were used. All array scans passed standard AFFYMETRIX QC.

Robust Multiarray algorithm (RMA) was used for preprocessing of raw signals (Irizarry et al, 2003. available at ncbi.nlm.nih.gov/pubmed/12925520). All probe sets available for the genes of interest were retrieved as reported below. For gene CD274, when several probe sets were available to represent this gene, the probe set with the probe set with the highest average expression value (defined as an arithmetical average of expression of a given probe set) was selected to represent the gene:

CD274 (PDL1)

223834_at selected for PDL1

227458_at

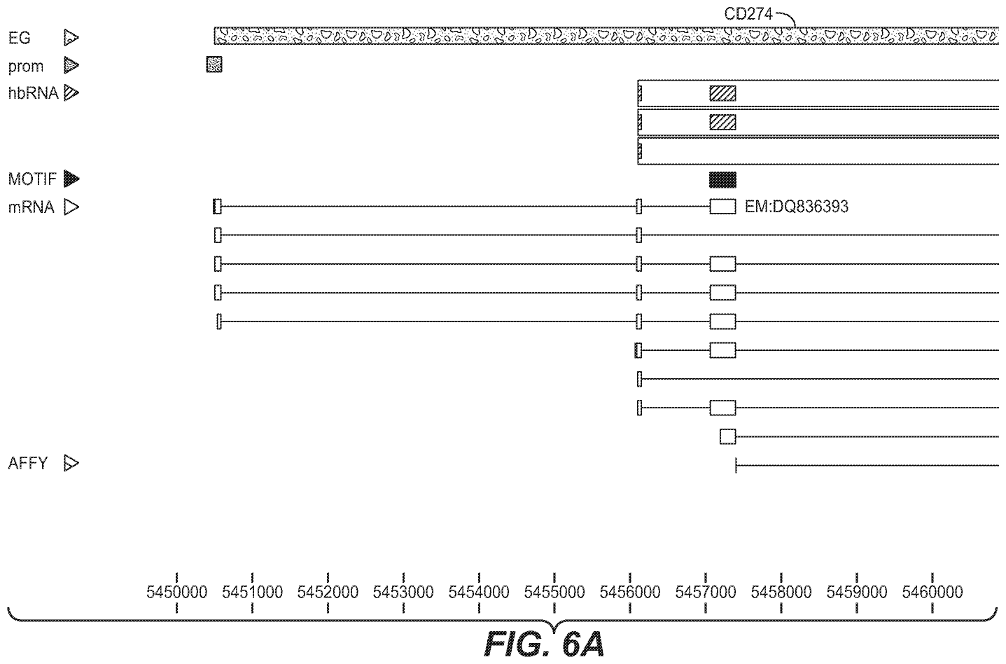

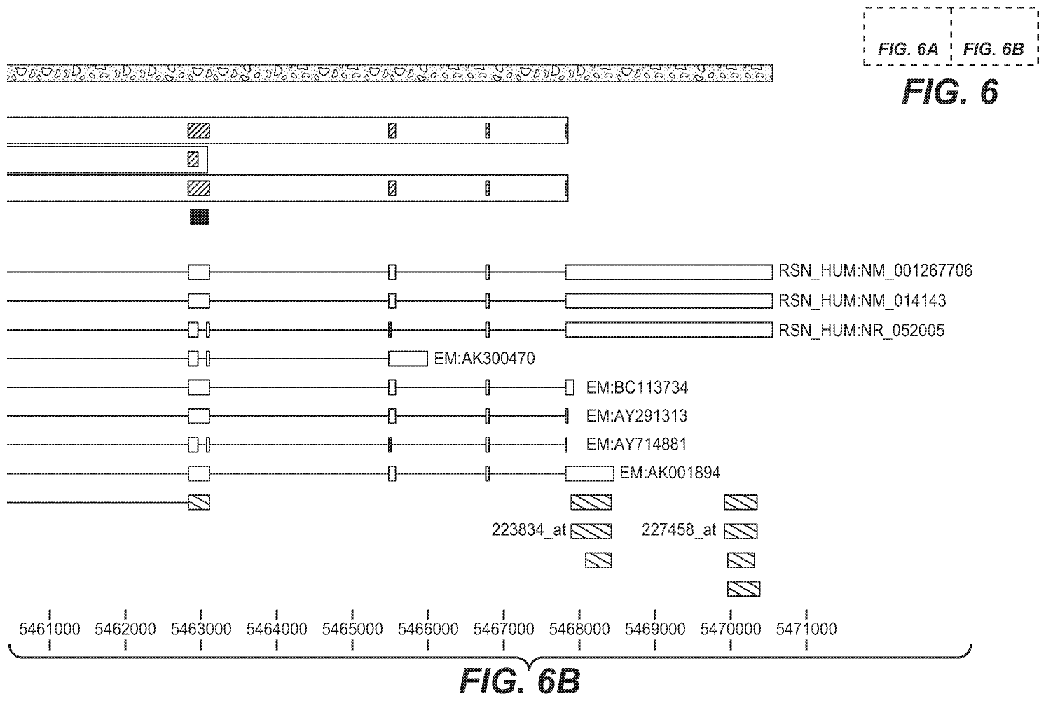

The selected probe set corresponds to the last exon/3′UTR of the gene and captures all known RefSEq mRNAs (see FIGS. 6A and 6B)

IFNG

210354_at

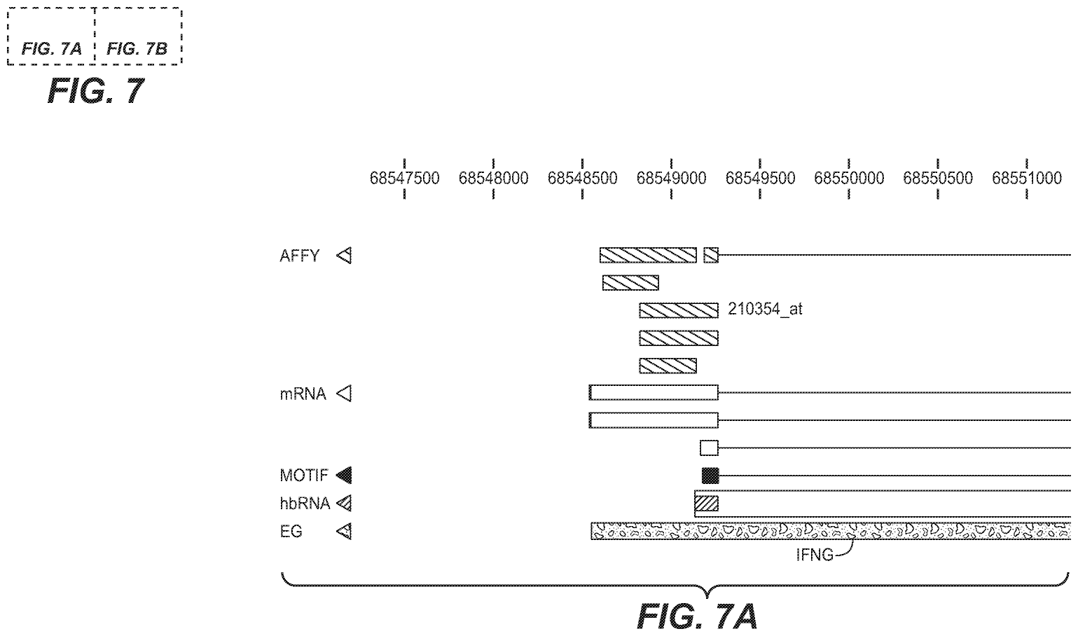

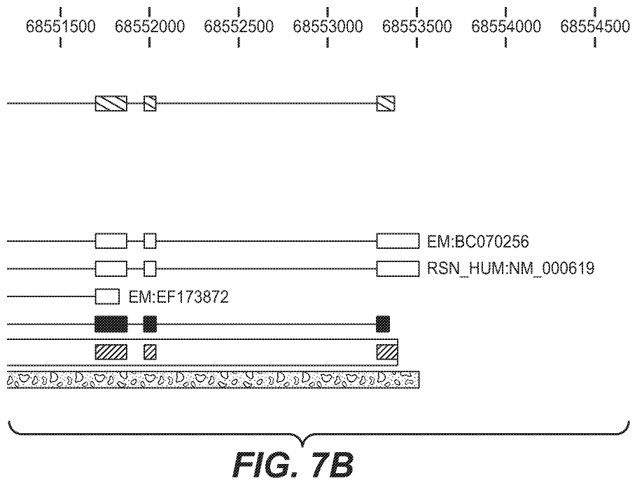

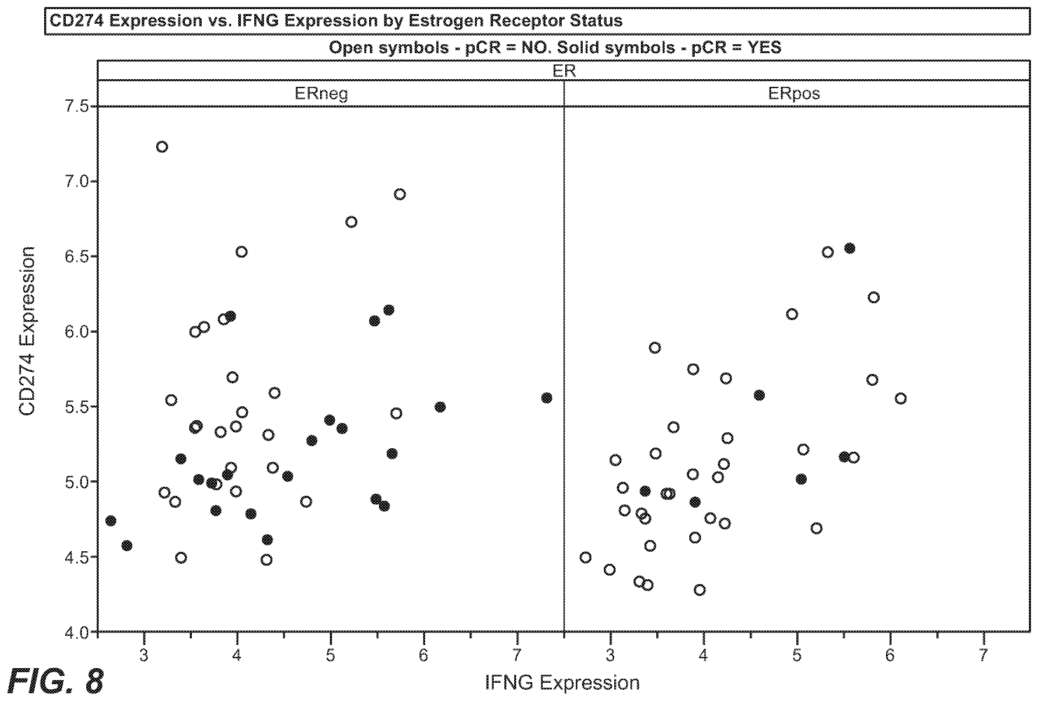

This probe set also represents the last exon/3′UTR of the gene and captures all known RefSEq mRNAs (see FIGS. 7A and 7B)

FIG. 8 shows joint distribution of the expression of the above genes in the samples of both ER− and ER− populations. Symbol types correspond to the final pCR status (solid: pCRachieved, open—pCR not achieved).

More details on distribution of CD274 and IFNG expression across ER and pCR strata can be found in Appendix I.

For every ER subpopulation, a logistic regression model was constructed that relates expression of the selected genes with clinical response adjusted for patient age, cancer type, and nodal status: Response˜Patient.Age+Cancer.Type+pN+CD274+IFNG

1. Er− Population.

Summarized model output is given below. Odds ratios are (OR) provided per unit change of biomarker value. As the expression values are given on log 2 scale, one unit change would correspond to 2-fold overexpression. For details see Appendix.

| | ER− population | |
|---|---|---|
| | | LR test |
| Term | OR (95% CI) | p-value |
| CD274 | 5.2 (1.5; 26.7) | 0.008 |
| IFNG | 0.30 (0.10; 0.74) | 0.007 |
| Patient | | 0.24 |
| Age | | |
| Cancer | | 0.91 |
| Type | | |
| pN | | 0.87 |

The final model for predicting probability for a particular patient to respond to the treatment includes expression of CD274 and IFNG and looks like:

p(pCR)=−3.737+1.607*CD274−1.069*IFNG

2. ER+ Population.

Summarized model output is given below. Odds ratios are (OR) provided per unit change of biomarker value. As the expression values are given on log 2 scale, one unit change would correspond to 2-fold overexpression. For details see Appendix.

| | ER+ population | |
|---|---|---|
| | | LR test |
| Term | OR (95% CI) | p-value |
| CD274 | | 0.93 |
| IFNG | | 0.23 |
| Patient | | 0.34 |
| Age | | |
| Cancer | | 0.39 |
| Type | | |
| pN | | 0.92 |

The role of PDL1 expression is evident in ER− subpopulation of HER2+ breast cancer patients that underwent combinational treatment with Trastuzumab and chemotherapy in the neoadjuvant setting. Namely, overexpression of PDL1 at diagnosis corresponds to a lower rate of response to neoadjuvant therapy (i.e. a lower rate of response to combinational treatment with Trastuzumab and chemotherapy). This holds irrespective of patient age, cancer type, or lymph node status. A baseline assessment of gene expression of either of the two biomarkers, PDL1 and INFG, respectively, allows to identify if a patient is likely to experience a greater benefit if a PDL-1 targeted therapy is added to Trastuzumab and chemotherapy.

The following relates to a cut-off value allowing determining a patient as being in need of a PD-L1 inhibitor cotherapy in accordance with the present invention.

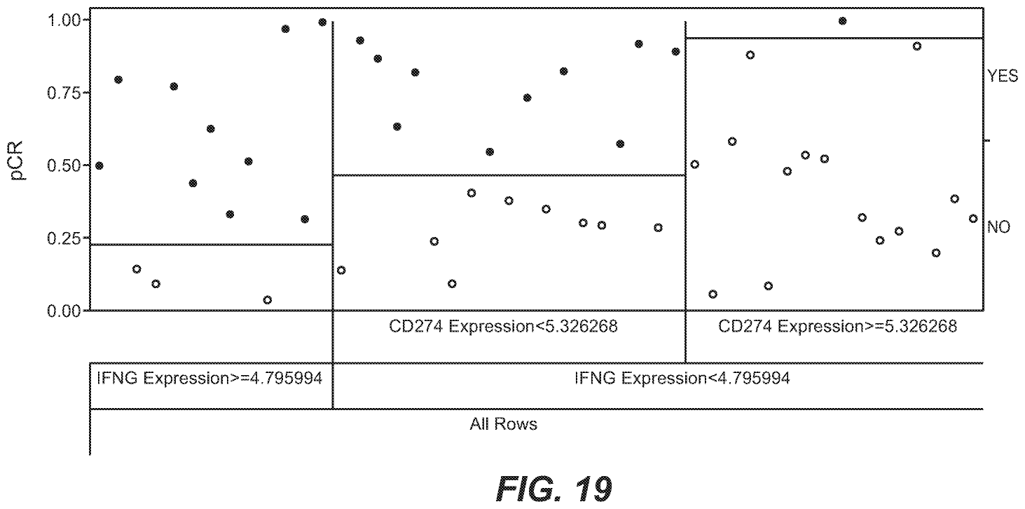

If a gene expression analysis gives a result for IFNG expression higher or equal to 4.8 no combination treatment (HER2-targeted and PDL1-targeted) is recommended and no further PDL1 assessment would be necessary. If a gene expression analysis gives a result for IFNG lower than 4.8 a parallel assessment of PDL-1 is necessary. If PDL-1 gene expression analysis then gives a result of higher or equal to 5.3 a combination treatment (HER2-targeted and PDL1-targeted) is recommended (see FIG. 19).

### APPENDIX I

ER− Subpopulation

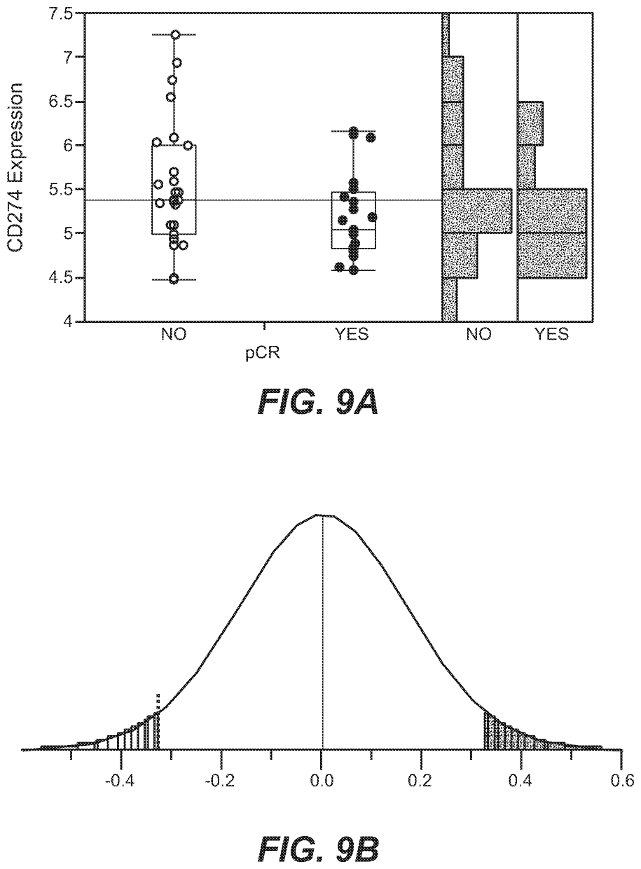

Oneway Analysis of CD274 Expression by pCR ER=ERneg (See FIG. 9A)

t Test

YES-NO

Assuming unequal variances

| | | |
|---|---|---|
| Difference | −0.32948 t Ratio | −1.94171 |
| Std Err Dif | 0.16969 DF | 45.11513 |
| Upper CL Dif | 0.01226 Prob > \|t\| | 0.0584 |
| Lower CL Dif | −0.67122 Prob > t | 0.9708 |
| Confidence | 0.95 Prob < t | 0.0292* |

The results are also shown in FIG. 9B.

Oneway Analysis of IFNG Expression by pCR ER ERneg

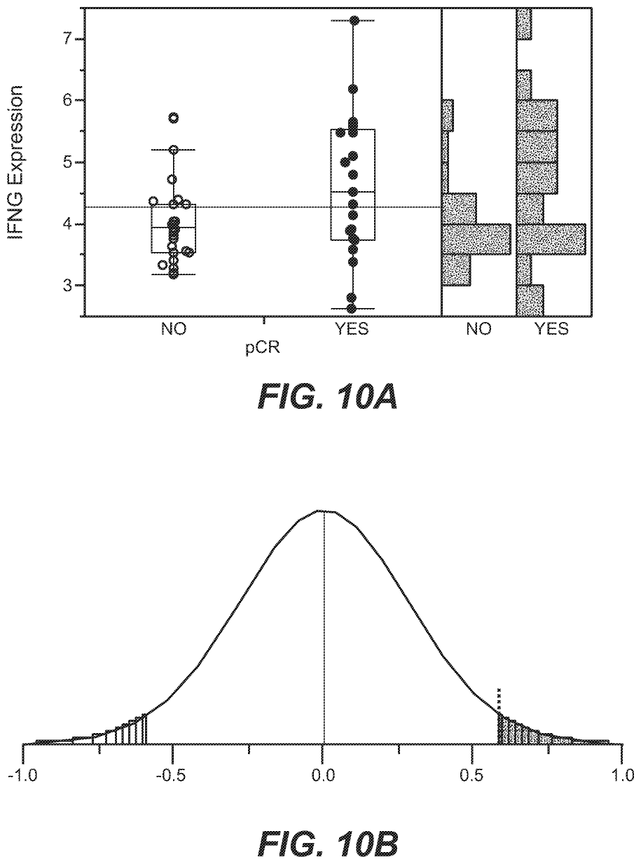

The results are shown in FIG. 10A.

t Test

YES-NO

Assuming unequal variances

| | | |
|---|---|---|
| Difference | 0.58405 t Ratio | 2.044225 |
| Std Err Dif | 0.28571 DF | 30.21429 |
| Upper CL Dif | 1.16737 Prob > \|t\| | 0.0497* |
| Lower CL Dif | 0.00073 Prob > t | 0.0249* |
| Confidence | 0.95 Prob < t | 0.9751 |

The results are shown in FIG. 10B.

ER+ Subpopulation

Oneway Analysis of CD274 Expression by pCR ER=ERpos

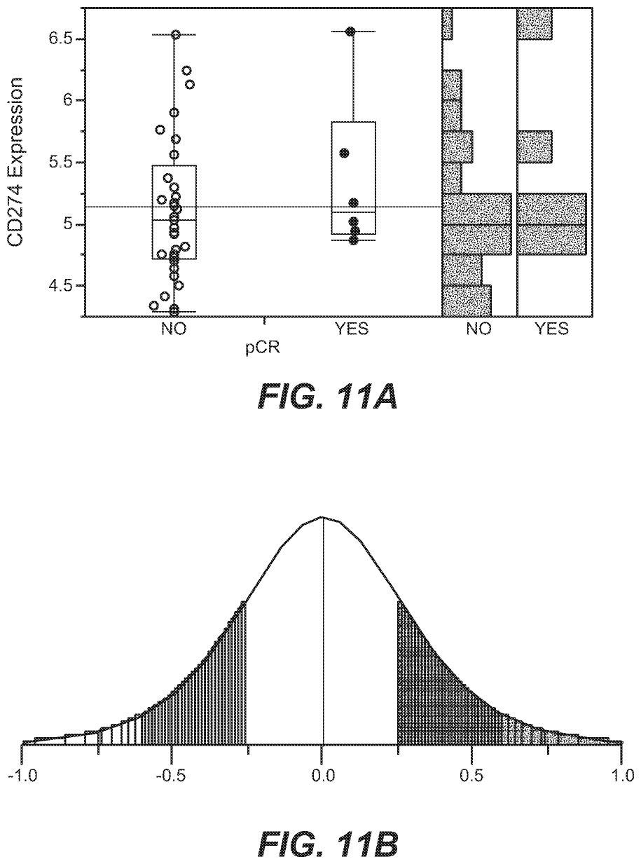

The results are shown in FIG. 11A.

t Test

YES-NO

Assuming unequal variances

| | | |
|---|---|---|
| Difference | 0.25169 t Ratio | 0.898709 |
| Std Err Dif | 0.28006 DF | 6.542171 |
| Upper CL Dif | 0.92345 Prob > \|t\| | 0.4007 |
| Lower CL Dif | −0.42006 Prob > t | 0.2003 |
| Confidence | 0.95 Prob < t | 0.7997 |

The results are shown in FIG. 11B.

Oneway Analysis of IFNG Expression by pCR ER=ERpos

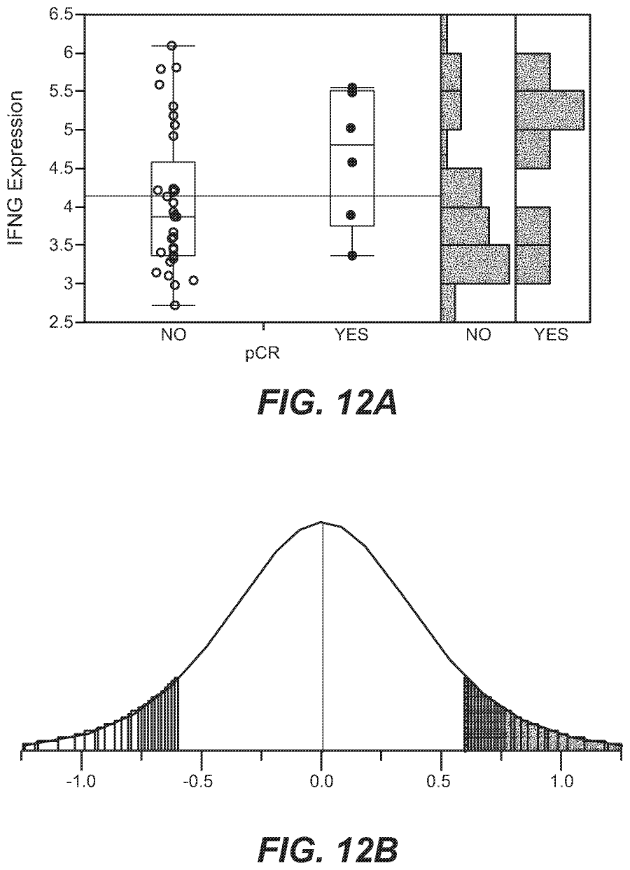

The results are shown in FIG. 12A.

t Test

YES-NO

Assuming unequal variances

| | | |
|---|---|---|
| Difference | 0.5931 t Ratio | 1.501336 |
| Std Err Dif | 0.3951 DF | 7.109044 |
| Upper CL Dif | 1.5244 Prob > \|t\| | 0.1763 |
| Lower CL Dif | −0.3382 Prob > t | 0.0882 |
| Confidence | 0.95 Prob < t | 0.9118 |

The results are shown in FIG. 12B.

### APPENDIX II

Nominal Logistic Fit for pCR ER=ERneg

Converged in Gradient, 5 iterations

Whole Model Test

| Model | −LogLikelihood | DF | Chi Square | Prob > ChiSq |
|---|---|---|---|---|
| Difference | 6.784783 | 6 | 13.56957 | 0.0348* |
| Full | 26.110299 | | | |
| Reduced | 32.895082 | | | |

| | |
|---|---|
| RSquare (U) | 0.2063 |
| AICc | 69.0206 |
| BIC | 79.319 |
| Observations (or Sum Wgts) | 48 |

| Measure | Training Definition |
|---|---|
| Entropy RSquare | 0.21063 1 − Loglike(model)/Loglike(0) |
| Generalized RSquare | 0.331 (1 − (L(0)/L(model))\hat{ }(2/n))/(1 − L(0)\hat{ }(2/n)) |
| Mean -Log P | 0.5440 Σ − Log(ρ[j])/n |
| RMSE | 0.4278 √ Σ(y[j] − ρ[j])²/n |
| Mean Abs Dev | 0.3665 Σ \|y[j] − ρ[j]\|/n |
| Misclassification Rate | 0.2292 Σ (ρ[j] ≠ ρMax)/n |
| N | 48 n |

Lack Of Fit

| Source | DF | −LogLikelihood | Chi Square |
|---|---|---|---|
| Lack Of Fit | 41 | 26.110299 | 52.2206 |
| Saturated | 47 | 0.000000 | Prob > ChiSq |
| Fitted | 6 | 26.110299 | 0.1125 |

Parameter Estimates

| Term | Estimate | Std Error | ChiSquare | Prob > CHiSq | Lower 95% | Upper 95% |
|---|---|---|---|---|---|---|
| Intercept | −5.9688255 | 4.1632695 | 2.06 | 0.1517 | −15.115329 | 1.70408281 |
| Patient Age | 0.04906238 | 0.0425045 | 1.33 | 0.2484 | −0.0324034 | 0.13829525 |
| Cancer Type[IBC] | −0.0943023 | 1.0982289 | 0.01 | 0.9316 | −2.5407977 | 2.23824618 |
| Cancer | −0.1514945 | 0.6544424 | 0.05 | 0.8169 | −1.5051269 | 1.21757158 |
| Type[LABC] | | | | | | |
| pN[N0] | 0.08157636 | 0.4979574 | 0.03 | 0.8699 | −0.8986622 | 1.09707358 |
| CD274 Expression | 1.64979222 | 0.7194762 | 5.26 | 0.0218* | 0.39533833 | 3.2836052 |
| IFNG Expression | −1.1882978 | 0.5122023 | 5.38 | 0.0203* | −2.3323039 | −0.2889168 |

For log odds of NO/YES

Effect Likelihood Ratio Tests

| Source | Nparm | DF | L-R ChiSquare | Prob > ChiSq |
|---|---|---|---|---|
| Patient Age | 1 | 1 | 1.38574446 | 0.2391 |
| Cancer Type | 2 | 2 | 0.19781033 | 0.9058 |
| pN | 1 | 1 | 0.02690704 | 0.8697 |
| CD274 Expression | 1 | 1 | 7.09800433 | 0.0077* |
| IFNG Expression | 1 | 1 | 7.15387723 | 0.0075* |

Odds Ratios
For pCR odds of NO versus YES

Tests and confidence intervals on odds ratios are likelihood ratio based.

Unit Odds Ratios

Per unit change in regressor

| Term | Odds Ratio | Lower 95% | Upper 95% | Reciprocal |
|---|---|---|---|---|
| Patient Age | 1.050286 | 0.968116 | 1.148315 | 0.9521217 |
| CD274 Expression | 5.205898 | 1.484886 | 26.67176 | 0.1920898 |
| IFNG Expression | 0.30474 | 0.097072 | 0.749074 | 3.2814908 |

Odds Ratios for Cancer Type

| Level1 | /Level2 | Odds Ratio | Prob > Chisq | Lower 95% | Upper 95% |
|---|---|---|---|---|---|
| LABC | IBC | 0.9444125 | 0.9722 | 0.0282989 | 35.902054 |
| OPERABLE | IBC | 1.405087 | 0.8471 | 0.0357479 | 68.159191 |
| OPERABLE | LABC | 1.4877895 | 0.6568 | 0.2518769 | 9.0216463 |
| IBC | LABC | 1.0588593 | 0.9722 | 0.0278536 | 35.337072 |
| IBC | OPERABLE | 0.7116997 | 0.8471 | 0.0146715 | 27.973694 |
| LABC | OPERABLE | 0.6721381 | 0.6568 | 0.1108445 | 3.9701934 |

Odds Ratios for pN

| Level1 | /Level2 | Odds Ratio | Prob > Chisq | Lower 95% | Upper 95% |
|---|---|---|---|---|---|
| N1 | N0 | 0.8494615 | 0.8697 | 0.1114536 | 6.033483 |
| N0 | N1 | 1.1772165 | 0.8697 | 0.1657417 | 8.9723459 |

Receiver Operating Characteristic

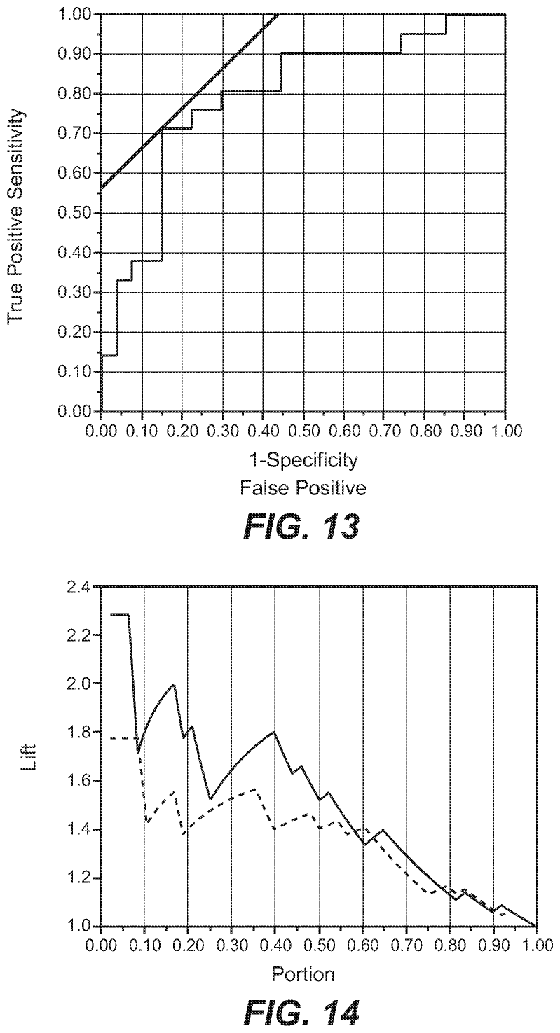

(see FIG. 13)

Using pCR=‘YES’ to be the positive level
AUC
0.79718

Confusion Matrix

Actual

Predicted

| Training | NO | YES |
|---|---|---|
| NO | 22 | 5 |
| YES | 6 | 15 |

Lift Curve (see FIG. 14)
pCR
NO
YES

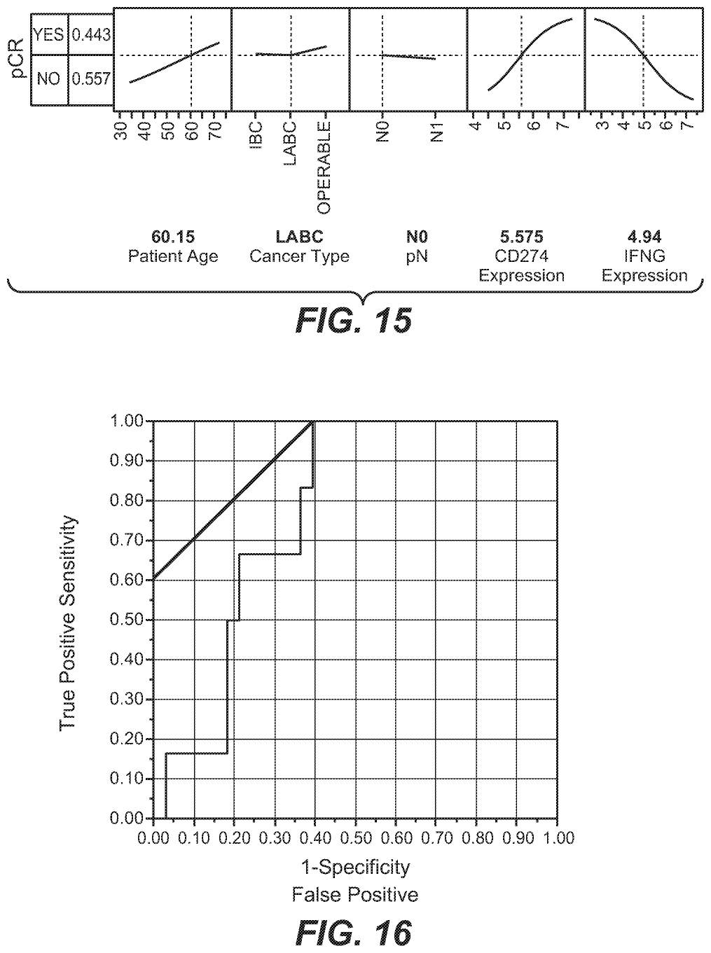

Prediction Profiler (see FIG. 15)

Nominal Logistic Fit for pCR ER = ERpos
Converged in Gradient, 19 iterations
Whole Model Test

| Model | -LogLikelihood | DF | ChiSquare | Prob > ChiSq |
|---|---|---|---|---|
| Difference | 2.400597 | 6 | 4.801193 | 0.5696 |
| Full | 14.343001 | | | |
| Reduced | 16.743598 | | | |
| RSquare (U) | 0.1434 | | | |
| AICc | 46.2989 | | | |
| BIC | 54.3309 | | | |
| Observations (or Sum Wgts) | 39 | | | |

| Measure | Training Definition |
|---|---|
| Entropy RSquare | 0.1434 1-Loglike(model)/Loglike(0) |
| Generalized RSquare | 0.2010 (1-(L(0)/L(model))\hat{ }(2/n))/(1-L(0)\hat{ }(2/n)) |
| Mean -Log p | 0.3678 Σ -Log(p[j])/n |
| RMSE | 0.3462 √ Σ(y[j] − ρ[j])²/n |
| Mean Abs Dev | 0.2351 Σ \|y[j] − ρ[j]}/n |
| Misclassification Rate | 0.1795 Σ (ρ[j] ≠ ρMax)/n |
| N | 39 n |

Lack Of Fit

| Source | DF | -LogLikelihood | ChiSquare |
|---|---|---|---|
| Lack Of Fit | 32 | 14.343001 | 28.686 |
| Saturated | 38 | 0.000000 | Prob > ChiSq |
| Fitted | 6 | 14.343001 | 0.6351 |

Parameter Estimates

| | | | | | Prob > | | |
|---|---|---|---|---|---|---|---|
| Term | | Estimate | Std Error | ChiSquare | ChiSq | Lower 95% | Upper 95% |
| Intercept | Unstable | 7.20306909 | 3597.5107 | 0.00 | 0.9984 | −7043.7884 | 7058.1945 |
| Patient Age | | 0.0578149 | 0.0628112 | 0.85 | 0.3573 | −0.0560483 | 0.19608254 |
| Cancer | Unstable | 12.0092513 | 7195.0139 | 0.00 | 0.9987 | −14089.959 | 14113.9773 |
| Type[IBC] | | | | | | | |
| Cancer | Unstable | −6.5864683 | 3597.507 | 0.00 | 0.9985 | −7057.5706 | 7044.39766 |
| Type[LABC] | | | | | | | |
| pN[N0] | | −0.0542869 | 0.5572904 | 0.01 | 0.9224 | −1.1698378 | 1.117206 |
| CD274 | | 0.08485271 | 0.9859164 | 0.01 | 0.9314 | −1.8704698 | 2.14104768 |
| Expression | | | | | | | |
| IFNG | | −0.7334678 | 0.6191817 | 1.40 | 0.2362 | −2.0476903 | 0.45985303 |
| Expression | | | | | | | |

For log odds of NO/YES
Effect Likelihood Ratio Tests

| Source | Nparm | DF | L-R ChiSquare | Prob > ChiSq |
|---|---|---|---|---|
| Patient Age | 1 | 1 | 0.92588732 | 0.3359 |
| Cancer Type | 2 | 2 | 1.89140212 | 0.3884 |
| pN | 1 | 1 | 0.00946444 | 0.9225 |
| CD274 Expression | 1 | 1 | 0.00742213 | 0.9313 |
| IFNG Expression | 1 | 1 | 1.45693945 | 0.2274 |

Odds Ratios
For pCR odds of NO versus YES

Tests and confidence intervals on odds ratios are likelihood ratio based.

Unit Odds Ratios

Per unit change in regressor

| Term | Odds Ratio | Lower 95% | Upper 95% | Reciprocal |
|---|---|---|---|---|
| Patient Age | 1.059519 | 0.945493 | 1.216627 | 0.9438246 |
| CD274 Expression | 1.088557 | 0.154051 | 8.508347 | 0.9186476 |
| IFNG Expression | 0.480241 | 0.129033 | 1.583841 | 2.0822891 |

Odds Ratios for Cancer Type

| Level1 | /Level2 | Odds Ratio | Prob > Chisq | Lower 95% | Upper 95% |
|---|---|---|---|---|---|
| LABC | IBC | 8.3942e−9 | 0.2128 | 0 | 5.1523961 |
| OPERABLE | IBC | 2.6876e−8 | 0.4499 | 0 | 20.868673 |
| OPERABLE | LABC | 3.2017112 | 0.3193 | 0.2999262 | 36.429388 |
| IBC | LABC | 119129251 | 0.2128 | 0.1940845 | . |
| IBC | OPERABLE | 37207993 | 0.4499 | 0.0479187 | . |
| LABC | OPERABLE | 0.312333 | 0.3193 | 0.0274504 | 3.3341535 |

Odds Ratios for pN

| Level1 | /Level2 | Odds Ratio | Prob > Chisq | Lower 95% | Upper 95% |
|---|---|---|---|---|---|
| N1 | N0 | 1.1146872 | 0.9225 | 0.1070551 | 10.377869 |
| N0 | N1 | 0.8971126 | 0.9225 | 0.0963589 | 9.3409878 |

Receiver Operating Characteristic

(see FIG. 16)

Using pCR=‘YES’ to be the positive level
AUC
0.77273

Confusion Matrix

Actual

Predicted

| Training | NO | YES |
|---|---|---|
| NO | 32 | 1 |
| YES | 6 | 0 |

The present invention refers to the following nucleotide and amino acid sequences:

The sequences provided herein are, inter alia, available in the NCBI database and disclosed in WO 2010/077634 and can be retrieved from world wide web at ncbi.nlm.nih.gov/sites/entrez?db=gene; Theses sequences also relate to annotated and modified sequences. The present invention also provides techniques and methods wherein homologous sequences, and variants of the concise sequences provided herein are used.

SEQ ID NOS: 1-21 define the anti-PD-L1 antibody to be used in accordance with the present invention. SEQ ID NOS: 1-21 are shown in the sequence listing.

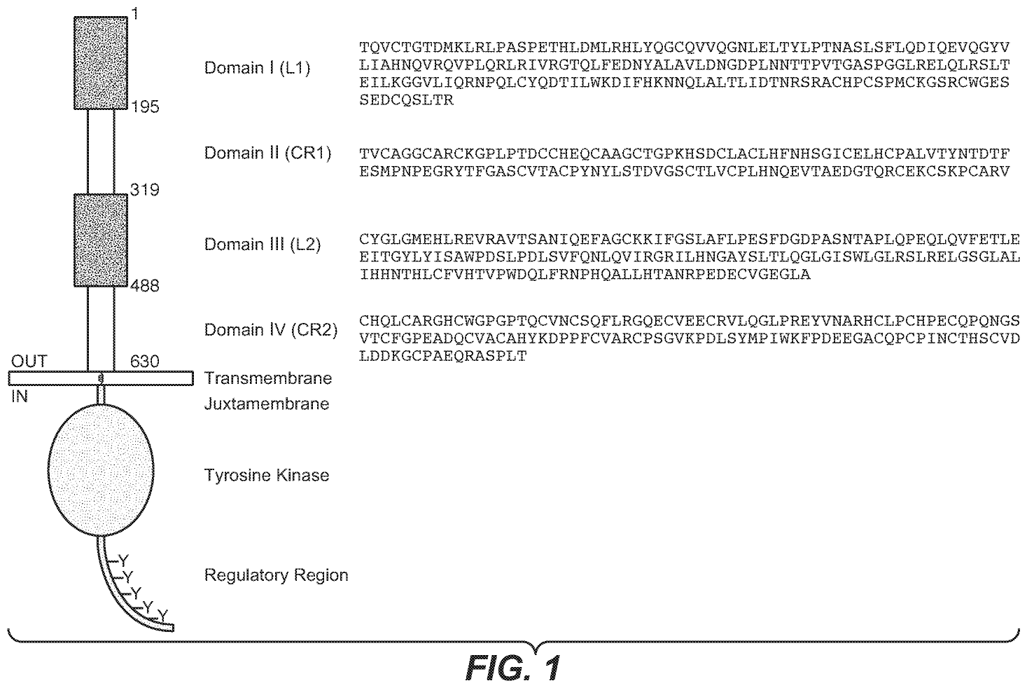

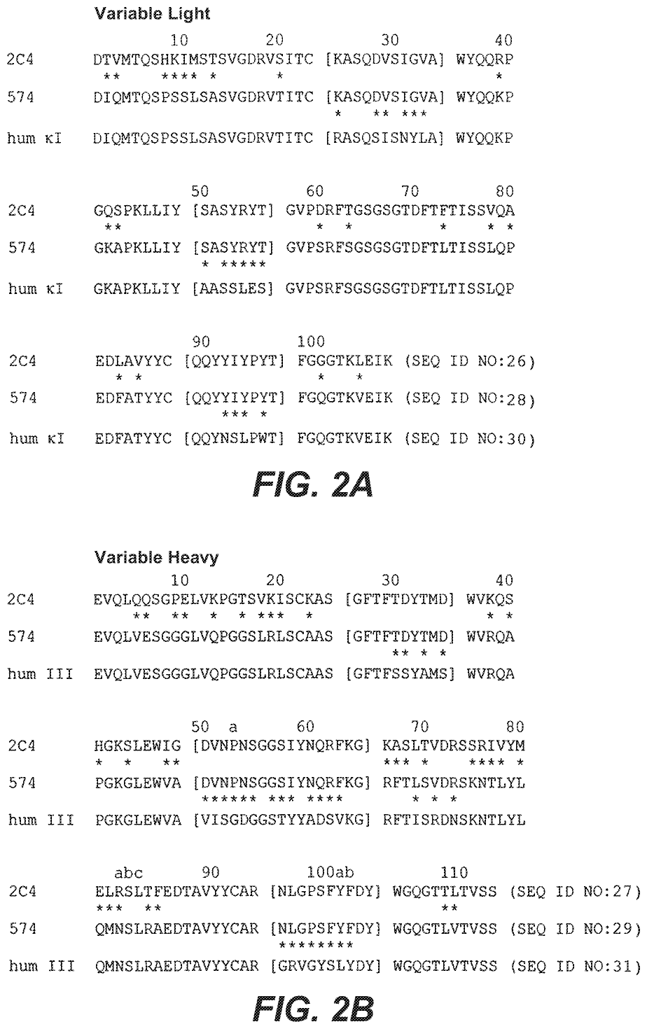

SEQ ID No. 22 to 37 show sequences of amino acid sequences for Domains I-IV of the HER2 protein (SEQ ID NO. 22-25, see also FIG. 1) and sequences of anti-HER2-antibodies. (SEQ ID NOS: 26 to 37; see also FIGS. 2A, 2B, 3A, 3B, 4A, 4B, 5A, and 5B).

SEQ ID No. 26:

Amino acid sequence of the variable

light (Vr) (FIG. 2A) domain of murine monoclonal antibody 2C4 (SEQ ID NOS: 26 and 27, respectively) as shown in FIGS. 2A and 2B.

SEQ ID No. 27:

Amino acid sequence of the variable heavy (V_{H}) (FIG. 2B) domain of murine monoclonal antibody 2C4 as shown in FIGS. 2A and 2B.

SEQ ID No. 28:

Amino acid sequence of the variable light (V_{L}) (FIG. 2A) domain of variant 574/Pertuzumab as shown in FIGS. 2A and 2B.

SEQ ID No. 29:

Amino acid sequence of the variable heavy (V_{H}) (FIG. 2B) domain of variant 574/Pertuzumab as shown in FIGS. 2A and 2B.

SEQ ID No. 30:

human V_{L} consensus frameworks (hum κ1, light kappa subgroup I; humIII, heavy subgroup III) as shown in FIGS. 2A and 2B.

SEQ ID No. 31:

human V_{H} consensus frameworks (hum κ1, light kappa subgroup I; humIII, heavy subgroup III) as shown in FIGS. 2A and 2B.

SEQ ID No. 32:

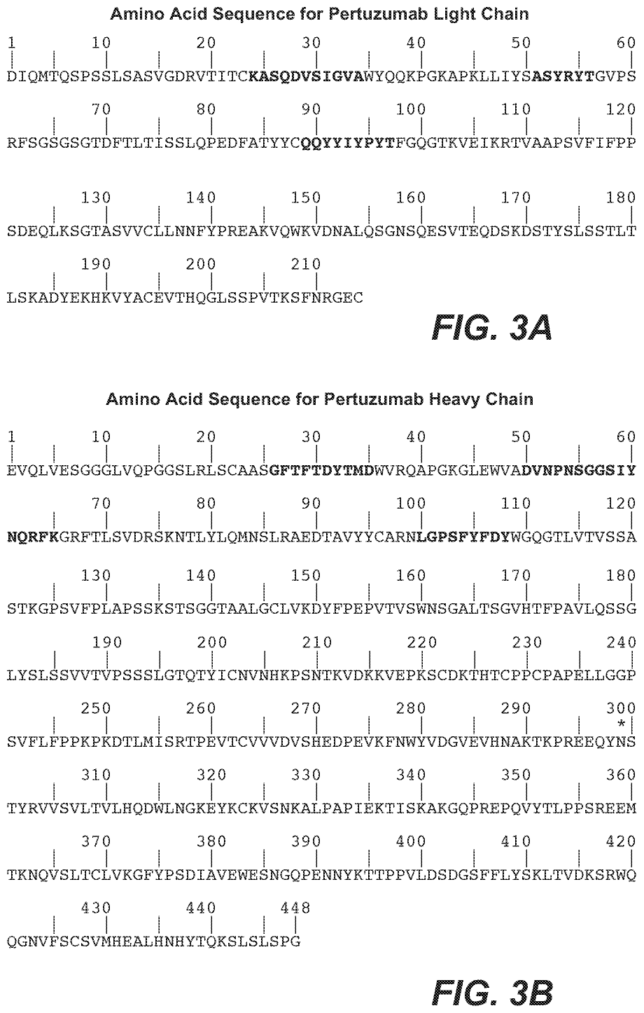

Amino acid sequences of Pertuzumab light chain as shown in FIG. 3A.

SEQ ID No. 33:

Amino acid sequences of Pertuzumab heavy chain as shown in FIG. 3B.

SEQ ID No. 34:

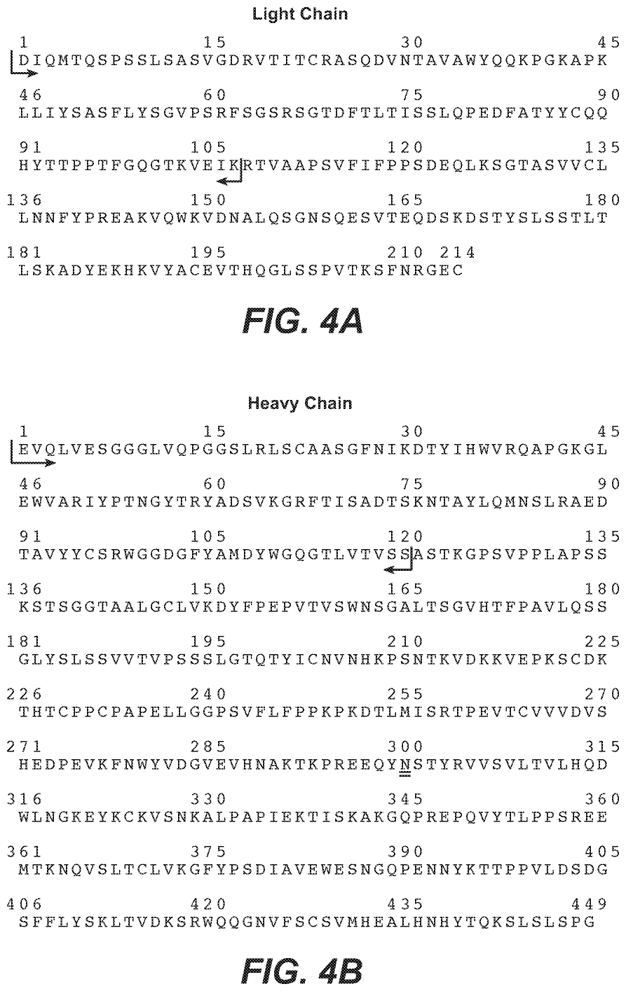

Amino acid sequence of Trastuzumab light chain domain as shown in FIG. 4A Boundaries of the variable light domain are indicated by arrows.

SEQ ID No. 35:

Amino acid sequence of Trastuzumab heavy chain as shown in FIG. 4B. Boundaries of the variable heavy domain are indicated by arrows.

SEQ ID No. 36:

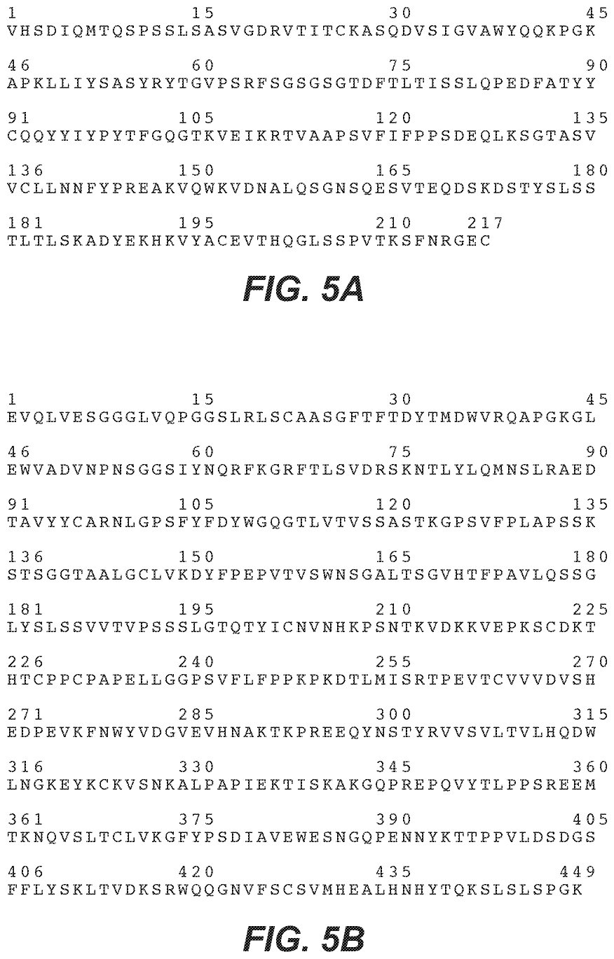

Amino acid sequence of variant Pertuzumab light chain sequence (FIG. 5A).

SEQ ID No. 37:

Amino acid sequence of variant Pertuzumab heavy chain sequence (FIG. 5B).

SEQ ID NO. 38:

Nucleotide sequence encoding Homo sapiens Progesterone Receptor (PR) NCBI Reference Sequence: NC_000011.9

>gi|224589802:c101000544-100900355 Homo sapiens chromosome 11, GRCh37.p10 Primary Assembly

SEQ ID No. 39:

Amino acid sequence of Homo sapiens Progesterone Receptor (PR)

PRGR_HUMAN Length: 933 Dec. 7, 2012 15:10 Type: P Check: 6067.

SEQ ID NO. 40:

Nucleotide sequence encoding Homo sapiens Estrogen Receptor (ER)

(NM_000125.3)

SEQ ID NO. 41:

Nucleotide sequence encoding Homo sapiens Estrogen Receptor (ER)

NCBI Reference Sequence: NC_000006.11

>gi|224589818:152011631-152424409 Homo sapiens chromosome 6, GRCh37.p10 Primary Assembly

SEQ ID No. 42:

Amino acid sequence of Homo sapiens Estrogen Receptor (ER)

>ENST00000206249_6

SEQ ID No. 43:

Nucleotide sequence encoding Homo sapiens programmed death ligand 1 (PD-L1)

NCBI Reference Sequence: NC_000009.11

>gi|224589821:5450503-5470567 Homo sapiens chromosome 9, GRCh37.p10 Primary Assembly

SEQ ID NO. 44

Nucleotide sequence encoding Homo sapiens programmed death ligand 1 (PD-L1) (CD274), transcript variant 1, mRNA

NCBI Reference Sequence: NM_014143.3

>gi|292658763|ref|NM_014143.31 Homo sapiens CD274 molecule (CD274), transcript variant 1, mRNA

SEQ ID No.45:

Amino acid sequence of Homo sapiens programmed death ligand 1 (PD-L1) (programmed cell death 1 ligand 1 isoform a precursor [Homo sapiens])

NCBI Reference Sequence: NP_054862.1

>gi|7661534|ref|NP_054862.11 programmed cell death 1 ligand 1 isoform a precursor [Homo sapiens]

SEQ ID No. 46:

Nucleotide sequence encoding Homo sapiens programmed death ligand 1 (PD-L1) (CD274), transcript variant 2, mRNA

NCBI Reference Sequence: NM_001267706.1

>gi|390979638|ref|NM_001267706.11 Homo sapiens CD274 molecule (CD274), transcript variant 2, mRNA

SEQ ID No. 47:

Amino acid sequence of Homo sapiens programmed death ligand 1 (PD-L1) (programmed cell death 1 ligand 1 isoform b precursor [Homo sapiens])

NCBI Reference Sequence: NP_001254635.1

>gi|390979639|ref|NP_001254635.11 programmed cell death 1 ligand 1 isoform b precursor [Homo sapiens]

SEQ ID No. 48:

Nucleotide sequence encoding Homo sapiens programmed death ligand 1 (PD-L1) (Homo sapiens CD274 molecule (CD274), transcript variant 3, non-coding RNA)

NCBI Reference Sequence: NR_052005.1

>gi|390979640|ref|NR 052005.11 Homo sapiens CD274 molecule (CD274), transcript variant 3, non-coding RNA

SEQ ID No. 49:

Nucleotide sequence encoding Homo sapiens interferon gamma (Homo sapiens chromosome 12, GRCh37.p10 Primary Assembly)

NCBI Reference Sequence: NC_000012.11

>gi|224589803:c68553521-68548550 Homo sapiens chromosome 12, GRCh37.p10 Primary Assembly

SEQ ID No. 50:

Nucleotide sequence encoding Homo sapiens interferon gamma, mRNA

NCBI Reference Sequence: NM_000619.2

>gi|56786137|ref|NM_000619.21 Homo sapiens interferon, gamma (IFNG), mRNA

SEQ ID No. 51:

Amino acid sequence of Homo sapiens interferon gamma, interferon gamma precursor [Homo sapiens]

NCBI Reference Sequence: NP_000610.2

>gi|56786138|ref|NP_000610.21 interferon gamma precursor [Homo sapiens]

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

1. A method of determining the need of a cancer patient for a PD-L1 inhibitor cotherapy, (i) wherein therapy comprising a modulator of the HER2/neu (ErbB2) signaling pathway and a chemotherapeutic agent is contemplated for the patient or (ii) wherein the patient is undergoing therapy comprising a modulator of the HER2/neu (ErbB2) signaling pathway and a chemotherapeutic agent, the method comprising the steps of
a) measuring in vitro in a sample from said patient the expression level of Estrogen receptor (ER) and of programmed death ligand 1 (PD-L1),
b) determining a patient as being in need of a PD-L1 inhibitor cotherapy if a low or absent ER expression level and an expression level of programmed death ligand 1 (PD-L1) that is increased in comparison to a control is measured in step (a).

2. A method of treating a cancer in a cancer patient for whom therapy comprising a modulator of the HER2/neu (ErbB2) signaling pathway and a chemotherapeutic agent is contemplated, the method comprising selecting a cancer patient whose cancer is determined to have a low or absent ER expression level and to have an increased expression level of programmed death ligand 1 (PD-L1) in comparison to a control, and administering to the patient an effective amount of a modulator of the HER2/neu (ErbB2) signaling pathway, of a chemotherapeutic agent and of a programmed death ligand 1 (PD-L1) inhibitor.

3. A method of treating a cancer in a cancer patient who is undergoing therapy comprising a modulator of the HER2/neu (ErbB2) signaling pathway and a chemotherapeutic agent, the method comprising selecting a cancer patient whose cancer is determined to have a low or absent ER expression level and to have an increased expression level of programmed death ligand 1 (PD-L1) in comparison to a control, and administering to the patient an effective amount of a programmed death ligand 1 (PD-L1) inhibitor.

4. The method of claim 1, further comprising measuring in vitro in a sample from said patient the expression level of interferon-gamma (IFNγ) and determining a patient as being in need of a PD-L1 inhibitor cotherapy if an expression level of interferon-gamma (IFNγ) that is decreased in comparison to a control is measured.

5. The method of claim 1; or the pharmaceutical composition of any one of claims 4, 5 and 7, wherein the ER expression level is ER(−).

6. The method of claim 1, wherein said modulator of the HER2/neu (ErbB2) signaling pathway is an inhibitor of HER shedding.

7. The method of claim 6, wherein said inhibitor of HER shedding is a HER2 shedding inhibitor.

8. The method of claim 6, wherein said inhibitor of HER shedding inhibits HER heterodimerization or HER homodimerization.

9. The method of claim 6, wherein said inhibitor of HER shedding is a HER antibody.

10. The method of claim 9, wherein said HER antibody binds to a HER receptor selected from the group consisting of EGFR, HER2 and HER3.

11. The method of claim 10, wherein said antibody binds to HER2.

12. The method of claim 11, wherein said HER2 antibody binds to sub-domain IV of the HER2 extracellular domain.

13. The method of claim 9, wherein said HER2 antibody is Herceptin/Trastuzumab.

14. The method of claim 1, wherein said modulator of the HER2/neu (ErbB2) signaling pathway is a HER dimerization/signaling inhibitor.

15. The method of claim 14, wherein said HER dimerization inhibitor is a HER2 dimerization inhibitor.

16. The method of claim 14, wherein said HER dimerization inhibitor inhibits HER heterodimerization or HER homodimerization.

17. The method of claim 14, wherein said HER dimerization inhibitor is a anti HER antibody.

18. The method of claim 17, wherein said HER antibody binds to a HER receptor selected from the group consisting of EGFR, HER2 and HER3.

19. The method of claim 18, wherein said antibody binds to HER2.

20. The method of claim 19, wherein said anti HER2 antibody binds to domain II of HER2 extracellular domain.

21. The method of claim 20, wherein said antibody binds to a junction between domains I, II and III of HER2 extracellular domain.

22. The method of claim 17, wherein said anti HER2 antibody is Pertuzumab.

23. The method of claim 1, wherein said chemotherapeutic agent is taxol or a taxol derivative.

24. The method of claim 23, wherein said taxol derivative is dodetaxel.

25. The method of claim 1, wherein said inhibitor of programmed death ligand 1 (PD-L1) is an antibody specifically binding to PD-L1 (anti-PD-L1 antibody).

26. The method of claim 25, wherein said antibody comprises an heavy chain variable region polypeptide comprising an HVR-H1, HVR-H2 and HVR-H3 sequence, wherein:
(SEQ ID NO: 1)
(a) the HVR-H1 sequence is GFTFSX1SWIH;
(SEQ ID NO: 2)
(b) the HVR-H2 sequence is AWIX2PYGGSX3YYADSVKG;
(SEQ ID NO: 3)
(c) the HVR-H3 sequence is RHWPGGFDY;
further wherein: X1 is D or G; X2 is S or L; X3 is T or S.

27. The method of claim 26, wherein X1 is D; X2 is S and X3 is T.

28. The method of claim 26, wherein said polypeptide further comprises variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4).

29. The method of claim 28, wherein the framework sequences are derived from human consensus framework sequences.

30. The method of claim 29, wherein the framework sequences are VH subgroup III consensus framework.

31. The method of claim 30, wherein one or more of the framework sequences is the following:
(SEQ ID NO: 4)
HC-FR1 is EVQLVESGGGLVQPGGSLRLSCAAS
(SEQ ID NO: 5)
HC-FR2 is WVRQAPGKGLEWV
(SEQ ID NO: 6)
HC-FR3 is RFTISADTSKNTAYLQMNSLRAEDTAVYYCAR
(SEQ ID NO: 7)
HC-FR4 is WGQGTLVTVSA.

32. The method of claim 26, wherein said heavy chain polypeptide is in combination with a variable region light chain comprising an HVR-L1, HVR-L2 and HVR-L3, wherein:
(SEQ ID NOs: 8)
(a) the HVR-L1 sequence is RASQX4X5X6TX7X8A;
(SEQ ID NOs: 9)
(b) the HVR-L2 sequence is SASX9LX10S,
and;
(SEQ ID NOs: 10)
(c) the HVR-L3 sequence is QQX11X12X13X14PX15T;
further wherein: X4 is D or V; X5 is V or I; X6 is S or N; X7 is A or F; X8 is V or L; X9 is F or T; X10 is Y or A; X11 is Y, G, F, or S; X12 is L, Y, F or W; X13 is Y, N, A, T, G, F or I; X14 is H, V, P, T or I; X15 is A, W, R, P or T.

33. The method of claim, wherein X4 is D; X5 is V; X6 is S; X7 is A; X8 is V; X9 is F; X10 is Y; X11 is Y; X12 is L; X13 is Y; X14 is H; X15 is A.

34. The method of claim 32, wherein said polypeptide further comprises variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4).

35. The method of claim 34, wherein the framework sequences are derived from human consensus framework sequences.

36. The method of claim 34, wherein the framework sequences are VL kappa I consensus framework.

37. The method of claim 36, wherein one or more of the framework sequences is the following:
(SEQ ID NO: 11)
LC-FR1 is DIQMTQSPSSLSASVGDRVTITC;
(SEQ ID NO: 12)
LC-FR2 is WYQQKPGKAPKLLIY;
(SEQ ID NO: 13)
LC-FR3 is GVPSRFSGSGSGTDFTLTISSLQPEDFATYYC;
(SEQ ID NO: 14)
LC-FR4 is FGQGTKVEIKR.

38. The method of claim 26, wherein said anti-PD-L1 antibody comprises a heavy chain and a light chain variable region sequence, wherein:
(a) the heavy chain comprises an HVR-H1, HVR-H2 and HVR-H3, wherein further:
(SEQ ID NO: 1)
(i) the HVR-H1 sequence is GFTFSX1SWIH;
(SEQ ID NO: 2)
(ii) the HVR-H2 sequence is AWIX2PYGGSX3YYADSVKG;
(SEQ ID NO: 3)
(iii) the HVR-H3 sequence is RHWPGGFDY,
and;
(b) the light chain comprises an HVR-L1, HVR-L2 and HVR-L3, wherein further:
(SEQ ID NOs: 8)
(iv) the HVR-L1 sequence is RASQX4X5X6TX7X8A;
(SEQ ID NOs: 9)
(v) the HVR-L2 sequence is SASX9LX10S;
(SEQ ID NOs: 10)
(vi) the HVR-L3 sequence is QQX11X12X13X14PX15T;
wherein: X1 is D or G; X2 is S or L; X3 is T or S; X4 may be D or V; X5 may be V or I; X6 may be S or N; X7 may be A or F; X8 may be V or L; X9 may be F or T; X10 may be Y or A; X11 may be Y, G, F, or S; X12 may be L, Y, F or W; X13 may be Y, N, A, T, G, F or I; X14 may be H, V, P, T or I; X15 may be A, W, R, P or T.

39. The method of claim 38, wherein X1 is D; X2 is S and X3 is T.

40. The method of claim 38, wherein X4=D, X5=V, X6=S, X7=A and X8=V, X9=F, and X10=Y, X11=Y, X12=L, X13=Y, X14=H and X15=A.

41. The method of claim 38, wherein X1=D, X2=S and X3=T, X4=D, X5=V, X6=S, X7=A and X8=V, X9=F, and X10=Y, X11=Y, X12=L, X13=Y, X14=H and X15=A.

42. The method of claim 38, wherein the antibody further comprises
(a) variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4), and
(b) variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4).

43. The method of claim 42, wherein the framework sequences are derived from human consensus framework sequences.

44. The method of claim 43, wherein the variable region heavy chain framework sequences are VH subgroup III consensus framework.

45. The method of claim 44, wherein one or more of the framework sequences is the following:
(SEQ ID NO: 4)
HC-FR1 is EVQLVESGGGLVQPGGSLRLSCAAS;
(SEQ ID NO: 5)
HC-FR2 is WVRQAPGKGLEWV;
(SEQ ID NO: 6)
HC-FR3 is RFTISADTSKNTAYLQMNSLRAEDTAVYYCAR;
(SEQ ID NO: 7)
HC-FR4 is WGQGTLVTVSA.

46. The method of claim 43, wherein the variable region light chain framework sequences are VL kappa I consensus framework.

47. The method of claim 46, wherein one or more of the framework sequences is the following:
(SEQ ID NO: 11)
LC-FR1 is DIQMTQSPSSLSASVGDRVTITC;
(SEQ ID NO: 12)
LC-FR2 is WYQQKPGKAPKLLIY;
(SEQ ID NO: 13)
LC-FR3 is GVPSRFSGSGSGTDFTLTISSLQPEDFATYYC,
and;
(SEQ ID NO: 14)
LC-FR4 is FGQGTKVEIKR.

48. The method of claim 43, wherein:
(a) the variable heavy chain framework sequences are the following:
(SEQ ID NO: 4)
(i) HC-FR1 is EVQLVESGGGLVQPGGSLRLSCAAS;
(SEQ ID NO: 5)
(ii) HC-FR2 is WVRQAPGKGLEWV;
(SEQ ID NO: 6)
(iii) HC-FR3 is RFTISADTSKNTAYLQMNSLRAEDTAVYYCAR;
(SEQ ID NO: 7)
(iv) HC-FR4 is WGQGTLVTVSA;
and;
(b) the variable light chain framework sequences are the following:
(SEQ ID NO: 11)
(i) LC-FR1 is DIQMTQSPSSLSASVGDRVTITC;
(SEQ ID NO: 12)
(ii) LC-FR2 is WYQQKPGKAPKLLIY;
(SEQ ID NO: 13)
(iii) LC-FR3 is GVPSRFSGSGSGTDFTLTISSLQPEDFATYYC;
(SEQ ID NO: 14)
(iv) LC-FR4 is FGQGTKVEIKR.

49. The method of claim, wherein the antibody further comprises a human constant region.

50. The method of claim 49, wherein the constant region is selected from the group consisting of IgG1, IgG2, IgG3 and IgG4.

51. The method of claim 50 wherein the constant region is IgG1.

52. The method of claim 48, wherein the antibody further comprises murine constant region.

53. The method of claim 52, wherein the constant region is selected from the group consisting of IgG1, IgG2A, IgG2B and IgG3.

54. The method of claim 53, wherein the constant region is IgG2A.

55. The method of claim 50, wherein said antibody has reduced or minimal effector function.

56. The method of claim 55, wherein the minimal effector function results from an effector-less Fc mutation.

57. The method of claim 56, wherein the effector-less Fc mutation is N297A.

58. The method of claim 56, wherein the effector-less Fc mutation is D265A/N297A.

59. Method of claim 55, wherein the minimal effector function results from aglycosylation.

60. The method of claim 26, wherein said antibody comprises a heavy chain and a light chain variable region sequence, wherein:
(a) the heavy chain comprises an HVR-H1, HVR-H2 and an HVR-H3, having at least 85% overall sequence identity to GFTFSDSWIH (SEQ ID NO:15), AWISPYGGSTYYADSVKG (SEQ ID NO:16) and RHWPGGFDY (SEQ ID NO:3), respectively, and
(b) the light chain comprises an HVR-L1, HVR-L2 and an HVR-L3, having at least 85% overall sequence identity to RASQDVSTAVA (SEQ ID NO:17), SASFLYS (SEQ ID NO:18) and QQYLYHPAT (SEQ ID NO:19), respectively.

61. The method of claim 60, wherein said sequence identity is at least 90%.

62. The method of claim 61, wherein said antibody further comprises:
(a) variable region heavy chain (VH) framework sequences juxtaposed between the HVRs according to the formula: (HC-FR1)-(HVR-H1)-(HC-FR2)-(HVR-H2)-(HC-FR3)-(HVR-H3)-(HC-FR4), and
(b) variable region light chain (VL) framework sequences juxtaposed between the HVRs according to the formula: (LC-FR1)-(HVR-L1)-(LC-FR2)-(HVR-L2)-(LC-FR3)-(HVR-L3)-(LC-FR4).

63. The method of claim 62, wherein said antibody further comprises a VH and VL framework region derived from a human consensus sequence.

64. The method of claim 63, wherein the VH framework sequence is derived from a Kabat subgroup I, II, or III sequence.

65. The method of claim 64, wherein the VH framework sequence is a Kabat subgroup III consensus framework sequence.

66. The method of claim 65, wherein the VH framework sequences are the following:
(SEQ ID NO: 4)
HC-FR1 is EVQLVESGGGLVQPGGSLRLSCAAS;
(SEQ ID NO: 5)
HC-FR2 is WVRQAPGKGLEWV;
(SEQ ID NO: 6)
HC-FR3 is RFTISADTSKNTAYLQMNSLRAEDTAVYYCAR;
(SEQ ID NO: 7)
HC-FR4 is WGQGTLVTVSA.

67. The method of claim 63, wherein the VL framework sequence is derived from a Kabat kappa I, II, III or IV subgroup sequence.

68. The method of claim 67, wherein the the VL framework sequence is a Kabat kappa I consensus framework sequence.

69. The method of claim 68, wherein the VL framework sequences are the following:
(SEQ ID NO: 11)
LC-FR1 is DIQMTQSPSSLSASVGDRVTITC;
(SEQ ID NO: 12)
LC-FR2 is WYQQKPGKAPKLLIY;
(SEQ ID NO: 13)
LC-FR3 is GVPSRFSGSGSGTDFTLTISSLQPEDFATYYC;
(SEQ ID NO: 14)
LC-FR4 is FGQGTKVEIKR.

70. The method of claim 26, wherein said antibody comprises a heavy chain and a light chain variable region sequence, wherein:
(SEQ ID NO: 20)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSA,
(a) the heavy chain sequence has at least 85% sequence identity to the heavy chain sequence:
and
(b) the light chain sequence has at least 85% sequence identity to the light chain sequence:
(SEQ ID NO: 21)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

71. The method of claim 70, wherein the sequence identity is at least 90%.

72. The method of claim 26, wherein said antibody comprises a heavy chain and light chain variable region sequence, wherein:
(a) the heavy chain comprises the sequence: EVQLVESGGGLVQPGGSLRLS CAASGFTFSDSWIHWVRQAPGKGLEWVAWISPYGGSTYYADSVKGRFTISADTSKNTAYL QMNSLRAEDTAVYYCARRHWPGGFDYWGQGTLVTVSA (SEQ ID NO:20), and
(b) the light chain comprises the sequence: DIQMTQSPSSLSASVGDRVTITC RASQDVSTAVAWYQQKPGKAPKLLIYSASFLYSGVPSRFSGSGSGTDFTLTISSLQPEDFAT YYCQQYLYHPATFGQGTKVEIKR (SEQ ID NO:21).

73. The method of claim 1, wherein said cancer is a solid cancer.

74. The method of claim 73, wherein said solid cancer is breast cancer or gastric cancer.

75. The method of claim 73, wherein said solid cancer is breast cancer.

79. The method of claim 1, wherein the expression level of PD-L1 is higher or equal to 5.3 determined by routine methods like Affymetrix.

76. The method claim 1, wherein the expression level of PD-L1 is the mRNA expression level.

77. The method of claim 76, wherein the mRNA expression level of PD-L1 is assessed by in situ hybridization, micro-arrays, or RealTime PCR.

78. The method of claim 1, wherein the expression level of PD-L1 is the protein expression level.

79. The method of claim 78, wherein said protein expression level of PD-L1 is assessed by immunoassay, gel- or blot-based methods, IHC, mass spectrometry, flow cytometry, or FACS.

80. The method of claim 1, wherein the patient to be treated is a human.

81. The method of claim 1, wherein said modulator of the HER2/neu (ErbB2) signaling pathway, said chemotherapeutic agent and said inhibitor of programmed death ligand 1 (PD-L1) are to be administered in a neoadjuvant setting or adjuvant setting or metastatic setting.

82. A method for treating cancer comprising administering an effective amount of a modulator of the HER2/neu (ErbB2) signaling pathway, a chemotherapeutic agent and an inhibitor of programmed death ligand 1 (PD-L1) to a subject in need thereof.
